(19) United States
(12) Patent Application Publication
(10) Pub. No.: US 2014/0194479 A1
(43) Pub. Date: Jul. 10, 2014

(54) COMPOSITIONS AND METHODS FOR TREATING MOOD DISORDERS

(71) Applicants: Claudia SCHMAUSS, Stamford, CT (US); Amir LEVINE, New York, NY (US)

(72) Inventors: Claudia SCHMAUSS, Stamford, CT (US); Amir LEVINE, New York, NY (US)

(21) Appl. No.: 14/207,791

(22) Filed: Mar. 13, 2014

Related U.S. Application Data
(63) Continuation-in-part of application No. PCT/US2012/054735, filed on Sep. 12, 2012.
(60) Provisional application No. 61/539,398, filed on Sep. 26, 2011.
(60) Provisional application No. 61/534,570, filed on Sep. 14, 2011.

(51) Int. Cl. A61K 31/167; A61K 31/19; A61K 31/4406; A61K 31/138

(52) U.S. Cl. CPC A61K 31/167; A61K 31/138; A61K 31/19; A61K 31/4406
USPC 514/357; 514/491; 514/651; 514/557

(57) ABSTRACT

The present invention provides, inter alia, methods for enhancing the anti-depressant efficacy of a selective serotonin re-uptake inhibitor (SSRI) in a patient being treated for a mood disorder. These methods include administering to a patient in need thereof a therapeutically effective amount of an SSRI and a therapeutically effective amount of a modulator of histone expression. Also provided are methods for identifying a patient population that suffers from a mood disorder that is more likely to respond to SSRI treatment. Further provided are compositions for treating or ameliorating the effects of a mood disorder.

### CROSS-REFERENCE TO RELATED APPLICATIONS

The present invention claims benefit to and is a continuation-in-part of international application no. PCT/US2012/054735 filed Sep. 12, 2012. The '735 application claims benefit to U.S. provisional application Ser. No. 61/539,398 filed Sep. 26, 2011 and No. 61/534,570 filed Sep. 14, 2011. The entire contents of the above applications are incorporated by reference.

### GOVERNMENT FUNDING

This invention was made with government support under grant nos. RO1 MH 078993 and 1R21 MH099251-01 from the National Institutes of Health. The government has certain rights in the invention.

### FIELD OF INVENTION

The present invention provides, inter alia, compositions and methods for treating mood disorders, such as e.g., depression.

### INCORPORATION BY REFERENCE OF SEQUENCE LISTING

This application contains references to amino acids and/or nucleic acid sequences that have been filed concurrently herewith as sequence listing text file “0366415.txt”, file size of 4 KB, created on Mar. 12, 2014. The aforementioned sequence listing is hereby incorporated by reference in its entirety pursuant to 37 C.F.R. §1.52(e)(5).

### BACKGROUND OF THE INVENTION

Early life stress is a prominent risk factor for several psychiatric illnesses, including mood and anxiety disorders (Holmes et al., 2005). More than 30% of mental disorders are directly related to early life stress (Afifi et al., 2008; Green et al., 2010). Early life stress (childhood abuse and neglect, loss of parents, or extreme poverty) occurs worldwide and cannot be eliminated. Hence, developing therapies that prevent the long-term consequences of early life stress is of utmost importance, and necessitates a better understanding of the mechanisms by which early life stress triggers long-lasting alterations in gene expression and behavior.

While early-life stress effects on adult psychopathology may depend upon genetic risk, it is thought that the nature of gene and environment interaction is a critical determinant of outcome. In animal studies on the effect of early life stress, inbred strains of mice proved particularly useful because of their natural genetic variability and their stable behavioral differences at baseline and after stress exposure. This is best documented for the isogenic strains C57BI/6 and Balb/c that differ not only in their sensitivity to early life and adult stress (Holmes et al., 2005; Millstein and Holmes, 2007), but also in their susceptibility to develop frontal cortical gene expression changes (Bhansali et al., 2007; Navailles et al., 2010; Schmauss et al., 2010) and deficits in cognitive functions governed by the frontal cortex (Mehta and Schmauss, 2011) after early life stress exposure. Originally, such differences were widely assumed to relate to genetic differences between both strains. However, it has now been shown that the behavioral traits of both strains (Francis et al., 2003) as well as the neuroendocrine abnormalities that result from early life stress exposure (Murgatroyd et al., 2009) are also influenced by epigenetic mechanisms. This has led to the hypothesis that the influence of early environmental factors on the chromatin structure of certain genes is critical in the development of stable changes in gene expression and behavior (Dulac, 2010).

Histone acetylation is a dynamic process that is controlled by the antagonistic actions of histone acetyltransferases and histone deacetylases (HDACs). The balance between the activities of these enzymes serves as a key regulatory mechanism for gene expression. Histone tail acetylation neutralizes the basic charge of lysine residues and, thereby, unfolds chromatin and almost invariantly activates gene transcription. HDACs remove acetyl groups of histone tails and they can silence transcriptional activity (Kouzarides, 2007; Haberland et al., 2009).

Antidepressant medication is the leading choice for the treatment of mood disorders, and most of these drugs are either tricyclic antidepressants or selective serotonin re-uptake inhibitors (SSRIs). The efficacy of these drugs differs substantially among patients. For example, a recent meta-analysis revealed that antidepressant effects are superior over placebo only in severely depressed patients, but effects in patients with mild or moderate depression are minimal or non-existent (Fournier et al., 2010), and patients with a history of early life stress are especially non-responsive to antidepressant drugs (Nemeroff et al., 2003).

Therefore, there is need for additional treatment options for mood disorders, such as depression. The present invention is directed to meeting this and other needs.

### SUMMARY OF THE INVENTION

The inventor has found that the efficacy of antidepressant drugs depends upon the individual epigenetic phenotype. As disclosed in more detail herein, a novel treatment regimen that involves stimulation of histone expression has been shown to enhance the efficacy of SSRIs in subjects with low antidepressant responsiveness to such drugs.

One embodiment of the present invention is a method for enhancing the anti-depressant efficacy of a selective serotonin re-uptake inhibitor (SSRI) in a patient being treated for a mood disorder. This method comprises administering to a patient in need thereof a therapeutically effective amount of an SSRI and a therapeutically effective amount of a modulator of histone expression.

Another embodiment of the present invention is a method for identifying a patient population that suffers from a mood disorder that is more likely to respond to SSRI treatment. This method comprises:
(a) obtaining a biological sample from the patient;
(b) testing the biological sample to determine whether it has a reduced HDAC activity compared to a control population; and
(c) administering an effective amount of an SSRI to the patient, optionally together with an effective amount HDACi if the biological sample of the patient population has a reduced HDAC activity compared to the control population.

Yet another embodiment of the present invention is a composition for treating or ameliorating the effects of a mood disorder. This composition comprises an effective amount of an SSRI, an HDACi, and a pharmaceutically acceptable carrier.

A further embodiment of the present invention is a method for preventing, treating or ameliorating the effects of age-related cognitive deficits in a patient with a history of early life stress. This method comprises administering to the patient in need thereof an effective amount of an HDACi.

An additional embodiment of the present invention is a composition for preventing, treating or ameliorating the effects of age-related cognitive deficits in a patient with a history of early life stress. This composition comprises an effective amount of an HDACi and a pharmaceutically acceptable carrier.

Yet another embodiment of the present invention is a method for identifying and treating a patient population that suffers from a mood disorder that is more likely to respond to a combination SSRI/HDACi treatment. This method comprises:
(a) treating a patient population with an SSRI;
(b) determining which patients in the patient population are not responding to or who have low responsiveness to the SSRI treatment in step (a); and
(c) to those patients determined to be not responding to or to have low responsiveness to the SSRI treatment, administering the SSRI and an HDACi in an amount effective to treat the mood disorder.

### BRIEF DESCRIPTION OF THE DRAWINGS

FIG. 1 shows altered forebrain neocortical HDAC mRNA expression in infant maternal separation (IMS) Balb/c mice. FIG. 1A is a panel of figures showing a comparison of HDAC mRNA expression in the forebrain neocortex of SFR and IMS Balb/c mice at postnatal day 21 (P21), postnatal day 28 (P28), and postnatal day 60 (P60). FIG. 1B is a panel of figures showing the HDAC mRNA expression in the hippocampus of standard facility rearing (SFR) and IMS Balb/c mice at P21, P28, and P60. FIG. 1C shows the HDAC mRNA expression in the forebrain neocortex of SFR Balb/c mice and Balb/c mice reared in isolation (IR) during adolescent development. mRNA expression levels were determined by real-time PCR. Data are mean±sem of 5 to 7 animals per group and were compared by two-tailed Student's t test. *p<0.03, **p<0.003, ***p<0.001.

FIG. 2 shows the expression of acetylated histone H4 protein at P21 and P60. Representative Western blots are shown that were probed with antibodies directed against the indicated histone H4 modifications. The bar graphs summarize results of densitometry measures of optical densities (OD) of enhanced luminescent signals that were normalized to corresponding ODs of GAPDH signals, which were used as a control. White bars: SFR controls; gray bars: IMS mice. Data are mean±sem of 5 animals per group and were compared by two-tailed Student's t test.

FIG. 3 shows HDAC mRNA expression in the forebrain neocortex of SFR and IMS C57BI/6 mice. FIG. 3A shows HDAC mRNA expression at P21. FIG. 3B shows HDAC mRNA expression at P60. FIG. 3C shows the expression of acetylated histone H4 proteins at P60. Data are mean±sem of 5-6 animals per group. mRNA expression levels were determined by real-time PCR. Two-tailed Student's t tests revealed no significant differences between groups.

FIG. 4 shows the effect of theophylline on histone acetylation in IMS Balb/c mice. FIG. 4A shows the effect of theophylline on histone H4K12 acetylation. A representative Western blot is shown on the left. FIG. 4B shows the corresponding effect on histone H4K5, K8, and K16 acetylation. FIG. 4C shows the effect of theophylline on total H3 and H4 protein and on acetylated H4K9 protein. FIG. 4D shows the effect on di- and trimethylated histone H3K9 and H3K4, respectively. The bar graphs show ODs of enhanced luminescent signals that were normalized to corresponding ODs of GAPDH signals. White bars: SFR controls; gray bars: IMS mice. Data are mean±sem of 4 animals per group and were compared by two-tailed Student's t test. tph=theophylline.

FIG. 5 shows the performance of SFR and IMS Balb/c mice and theophylline-treated IMS Balb/c mice in the Elevated Plus Maze (EPM) (FIG. 5A) and the Force Swim Test (FST) (FIG. 5B). Data are mean±sem of 8 to 10 animals per group and were compared by one-way ANOVA. Statistical differences were resolved post hoc using Tukey-Kramer Multiple Comparisons tests as indicated. For the EPM results shown in FIG. 5A, the total number of open and closed arm entries are illustrated in the graph and the corresponding percentages of open arm entries are listed underneath the graph.

FIG. 6 shows the effect of adolescent SAHA and fluoxetine treatment on the performance of SFR Balb/c mice in the EPM (FIG. 6A) and FST (FIG. 6B). Data are mean±sem of 5 to 7 animals per group and were compared by one-way ANOVA. Statistical differences were resolved post hoc using Tukey-Kramer Multiple Comparisons tests as indicated. For the EPM results shown in FIG. 6A, the total number of open and closed arm crossings are illustrated in the graph and the corresponding percentages of open arm entries are listed underneath the graph. Note that SAHA-treated SFR mice exhibit the lowest percentage of open arm entries. When compared to non-treated SFR mice, this difference is significant (two-tailed Student's t test, p<0.04). tph=theophylline, fluox=fluoxetine.

FIGS. 7A and 7B show that in SFR mice, adolescent fluoxetine treatment does not alter histone H3/H4 expression. White bars: vehicle; gray bars: fluoxetine (16 mg/kg/day). (n=5/group).

FIG. 8 shows that co-treatment of SFR mice with SAHA and fluoxetine during adolescence increased expression of total histone H4, aCH4K5, and aCH4K12 protein. Data (mean±sem; n=5/group) were compared by two-tailed Student's t tests. fluox: fluoxetine.

FIG. 9 shows that co-treatment with SAHA and tianeptine does not significantly alter histone expression in SFR Balb/c mice. tia=tianeptine (10 mg/kg/day). Data (mean±sem; n=5/group) were compared by two-tailed Student's t tests.

FIG. 10 shows social recognition memory deficits in aging IMS Balb/c mice. 2 mo=2 months; 4 mo=4 months; 5 mo=5 months. In the SR test, the first measure (T1) counts the interaction time of the adult during a 5-minute exposure to an unfamiliar juvenile. After 2 hours, the test mouse is re-exposed to the same juvenile for minutes, and the time of interaction is recorded again (T2). The difference between T1 and T2 (T1-T2) is a measure of recognition memory, i.e., intact recognition memory reduces the T2 values. IMS Balb/c (but not IMS C57BI/6) mice exhibit deficits in recognition memory at 5 months of age. Data (mean±sem; n=8 mice/group) were compared by two-tailed Student's t tests.

FIG. 11 shows declining histone expression in IMS Balb/c mice during aging. FIG. 11A shows a representative Western blot indicating total histone H4 expression in SFR and IMS Balb/c mice at 2, 4, and 6 months of age. FIGS. 11B and 11C show that optical density (OD) measures revealed significantly decreased total histone H4 expression in IMS Balb/c mice, beginning at 4 months of age. Moreover, although the expression of aCH4K12 is significantly higher in IMS mice at 2 months of age, its expression is significantly lower at 4 and 6 months compared to SFR controls. Data (mean±sem; n=4-5 mice/group) were compared by two-tailed Student's t tests.

FIG. 12 shows chromatin fractionation of histone H4 protein. Free and chromatin-bound (cb) proteins were extracted using the chromatin fractionation protocol of Wysocka et al (2001) with one modification: the final chromatin pellet (P3) was re-suspended in 5 mM HEPES, 1.5 mM MgCl₂, 0.5 mM DTT, 26% glycerol, and 0.3 M NaCl, incubated on ice for 30 minutes, and centrifuged at 24,500 rpm. The respective supernatants were processed for Western blotting. Note that the free fraction of H4 protein is lower than the chromatin-bound fraction.

FIG. 13 shows that adult IMS Balb/c mice exhibit increased anxiety- and depression-like behavior, decreased HDAC expression, and increased acetylation of histone H4 proteins in the forebrain neocortex. Adolescent theophylline treatment led to reduced (−) aCH4K12 expression in adulthood and potentiated (+) the severity of the emotional phenotype of IMS mice. Increased aCH4K12 enrichment at the Gaq promotor leads to increased Gaq mRNA expression.

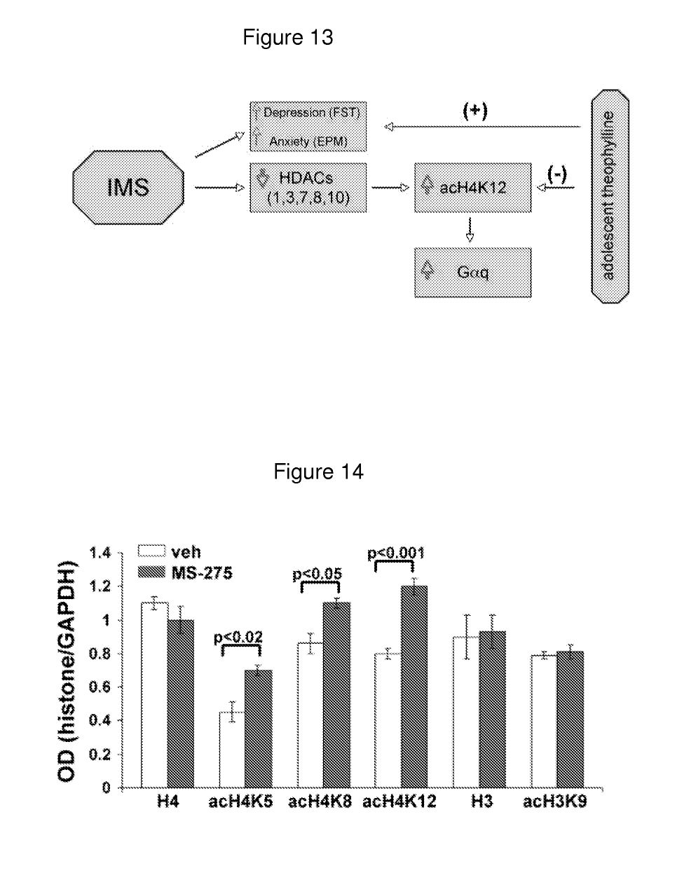

FIG. 14 shows that MS-275-treated SFR mice express increased aCH4 protein in the forebrain neocortex. Data are mean±sem (n=5/group) and were compared with two-tailed Student's t tests.

FIGS. 15A-D show the effect of adolescent fluoxetine with or without co-treatment of MS-275 or sodium butyrate (NaB) on the behavior of Balb/c mice in the forced swim test (FST) (FIG. 15A), the enrichment of aCH4K12 (FIG. 15B) and Pol II (FIG. 15C) at BDNF promotors 1 to 3, and on the transcription rates of BDNF mRNA variants 1 to 3 (FIG. 15D). Data in (FIG. 15A) are mean±sem of 8 animals per group. Data in (FIG. 15B) and (FIG. 15C) were obtained from ChIP/real-time PCR measures (mean±sem; n=4/group) using antibodies directed against aCH4K12 (FIG. 15B) and Pol II (FIG. 15C). PCR primers used for promotor-specific PCR amplifications are identical to those reported by Tsankova et al. (2006). Data in (FIG. 15D) are from RT-real-time PCR measures of BDNF mRNA levels (mean±sem, n=5/group). The PCR primers used are identical to those described by Tsankova et al. (2006). All data were compared by ANOVA and significant differences between groups were resolved post hoc using Tukey Kramer multiple comparisons as indicated. In all studies, male and female mice were used. No significant sex differences were identified.

### DETAILED DESCRIPTION OF THE INVENTION

One embodiment of the present invention is a method for enhancing the anti-depressant efficacy of a selective serotonin re-uptake inhibitor (SSRI) in a patient being treated for a mood disorder. This method comprises administering to a patient in need thereof a therapeutically effective amount of an SSRI and a therapeutically effective amount of a modulator of histone expression.

As used herein, a “mood disorder” means a group of diagnoses in the Diagnostic and Statistical Manual of Mental Disorders classification system where a disturbance in the person's mood is hypothesized to be the main underlying feature. The classification is also known as mood (affective) disorders in, e.g., International Classification of Diseases. Mood disorders include without limitation, depressive disorders, bipolar disorders, substance induced mood disorders, such as alcohol induced mood disorders, benzodiazepine induced mood disorders, and interferon-alpha induced mood disorders. Depressive disorders include major depressive disorder (MDD, commonly called major depression, unipolar depression, or clinical depression), disthymia, and depressive Disorder Not Otherwise Specified (DD-NOS). There are several subtypes of major depressive disorders, which include atypical depression (AD), melancholic depression, psychotic major depression (PMD, also known as psychotic depression), catatonic depression, postpartum depression (PPD), and seasonal affective disorder (SAD).

As used herein, a “patient” is a mammal, preferably, a human. The patient may be chronically stressed. The patient may also have a history of early life stress. As used herein, “chronically” stressed means being stressed for a prolonged period of time. Stress is any uncomfortable emotional experience accompanied by associated biochemical, physiological and behavioral changes. Chronic stress may occur in response to everyday stressors that are ignored or poorly managed, as well as to exposure to traumatic events. As used herein, “early life stress” means having had an acute or chronic stressor or traumatic event in childhood. Such stressors or events include, without limitation, natural disasters (flooding, fires, earthquakes, etc.) and also those that are man-made (assaults, motor-vehicle accidents, abuse, such as physical and sexual abuse, witnessing violence, etc.). Other non-limiting examples of early life stress include neglect, loss of parents, or extreme poverty in childhood.

As used herein, a “modulator” of histone expression means any agent that alters the function of or expression level of a protein, such as a histone deacetylase (HDAC) inhibitor, that results in the change in the expression level of histones or their post-translational modifications. Such alterations may be lowering or increasing the expression level of a protein, such as an HDAC, (either at the transcription stage or the translation stage), altering the sequence of a protein (by, e.g., mutation, pre-translational or post-translational modification or otherwise), or inhibiting or activating a protein (by, e.g., binding, phosphorylation, glycosylation, translocation or otherwise). Such alterations may be achieved genetically or pharmacologically.

An “HDAC” means the group of enzymes that remove acetyl groups (O═C—CH₃) from an ε-N-acetyl lysine amino acid on a histone. There are at least four classes of HDACs: I, II, III, and IV.

Preferably, the modulator of histone expression is a class I histone deacetylase inhibitor (HDACi). A “class I HDAC” means a phylogenetic class of HDACs that share domains with similarity to the yeast (Saccharomyces cerevisia) transcriptional regulator RPD3. Representative non-limiting members of the HDAC class I family include HDAC1, 2, 3, and 8, more particularly HDAC 1, 3, and 8, and preferably, HDAC 1 and 3. As used herein, “inhibit” and “inhibiting” and like terms, when used with respect to HDAC, means a decrease of the function of HDAC as a repressor of gene transcription. Non-limiting examples of how the function of HDAC may be decreased include decreasing expression of HDAC and altering the location of HDAC (e.g., from cytosolic to nuclear and vice versa).

Preferably, the HDACi is a selective inhibitor of HDAC class I. More preferably, the HDACi selectively inhibits HDAC 1, 3, 8, and combinations thereof, preferably HDAC 1 and 3. Non-limiting examples of HDACi according to the present invention include trichostatin A (TSA), suberoylanilide hydroxamic acid (SAHA), MS-275, pyroxamide, azelaic-1-hydroxamate-9-anilide (AAHA), CRA-024781 (Pharmacyclics, Sunnyvale, Calif.), bombesin-2 (BB2) receptor antagonist, JNJ-16241199 (Johnson & Johnson, Langhorne, Pa.), Oxamflatin, CG-1521 (Errant Gene Therapeutics, LLC, Chicago, Ill.), CG-1255 (Errant Gene Therapeutics, LLC, Chicago, Ill.), SK-7068 (In2Gen/SK Chemical Co., Suweon, Korea), SK-7041 (In2Gen/SK Chemical Co., Suweon, Korea), m-carboxycinnamic acid bis-hydroxamide (CBHA), Scriptaid (N-Hydroxy-1,3-dioxo-1H-benz[de]isoquinoline-2(3H)-hexan amide), SB-623 (Merrion Research I Limited, National Digital Park, Ireland), SB-639 (Merrion Research I Limited, National Digital Park, Ireland), SB-624 (Merrion Research I Limited, National Digital Park, Ireland), Panobinostat (LBH-589) (Novartis, Basel, Switzerland), NVP-LAQ824 (Novartis, Basel, Switzerland), butyrate, phenylbutyrate, valporic acid (VPA), Pivanex™ (Titan Pharmaceuticals, Inc.), AN-1 (Titan Pharmaceuticals, Inc.), tributyrin, compound G1, pivaloyloxymethyl butyrate, hyaluronic acid butyric acid ester (HA-But), Apicidine, Trapoxin-A, Trapoxin-B, cyclic hydroxamic acid-containing peptide 1 (CHAP-1), CHAP-31, CHAP-15, chlamidocin, HC-Toxin, WF-27082B (Fujisawa Pharmaceutical Company, Ltd., Osaka, Japan), Romidepsin (Gloucester Pharmaceuticals, Cambridge, Mass.), Spiruchostatin A, Depudesin, compound D1, Triacetylshikimic acid, Cyclostellettamine FFF1, Cyclostellettamine FFF2, Cyclostellettamine FFF3, Cyclostellettamine FFF4, MS-27-275 (Schering AG, Germany), Tacedinaline (N-acetyldinaline), ITF-2357 (Italfarmaco, Cinisello Balsamo, Italy), N-hydroxy-4-(3-methyl-2-phenyl-butyrylamino)benzamide (HDAC-42), MGCD-0103 (MethylGene Inc., Montreal, Quebec, Canada), PX-117794 (TopoTarget AS, Kobenhavn, Denmark), Belinostat (TopoTarget AS, Kobenhavn, Denmark), sulfonamide hydroxamic acid, mocetinostat (MethylGene), Entinostat (MS-275, Bayer AG), MG-2856 (MethylGene), MG-4230 (MethylGene), MG-4915 (MethylGene), MG-5026 (MethylGene), belinostat (TopoTarget AS), abexinostat (Celera), panobinostat (Novartis), CG-200745 (CrystalGenomi), SB-939 (S*BIO), chidamide (HUYA Bioscience), CHR-3996 (Chroma Therapeutics), AR-42 (Arno Therapeutics), RG-2833 (RepliGen), OCID-4681-S-01, (Orchid Pharmaceuticals), PCI-34051 (Pharmalcyclics), DAC-60 (Genextra), KAR-2581 (Karus Therapeutics), resminostat (Nycomed Pharma), 4SC-202 (Nycomed Pharma), YM-753 (Astellas), ACY-1216 (Acetylon), KAR-3000 (Karus Therapeutics), CU-906 (Curis), IKH-02 (IkerChem), ACY-257 (Acetylon), HDAC3 inhibitors (RepliGen), KAR-3166 (Karus Therapeutics), ONCO-101 (Oncoholdings), GSK424887 (GlaxoSmithKline), AGO178 (Novartis), TC5214 (AstraZeneca), pharmaceutically acceptable salts thereof, or combinations thereof. Preferably, the HDACi is SAHA, VPA, pharmaceutically acceptable salts thereof, or combinations thereof. Also preferably, the HDACi is Entinostat (MS-275, Bayer AG) or a pharmaceutically acceptable salt thereof.

Non-limiting examples of SSRIs according to the present invention include fluvoxamine, trazodone, indeloxazine, viloxazine, dapoxetine, duloxetin, fluoxetine, olanzapine, tramadol, paroxetine, tramadol, paroxetine mesylate, venlafaxine, citalopram, escitalopram, demexiptiline, vilazodone, nitroxazepine, desvenlafaxine, sertraline, venlafaxine, milnacipran, minaprine, quinupramine, amine transporter inhibitor (AMRI), venlafaxine (Auspex), DSP-1053 (Dainippon Sumitomo Pharma), SEP-228432 (Dainippon Sumitomo Pharma), DA-8031 (DA-8031), escitalopram (Lundbeck), NSD-788 (Neurosearch), SKL-10406 (SK Holdings), BMS-820836 (AMRI), pipamperone, doxepin (Winston Pharmaceuticals), levomilnacipran, clonazepam (Zydus) pharmaceutically acceptable salts thereof, or combinations thereof. Preferably, the SSRI is fluoxetine, pharmaceutically acceptable salts thereof, or combinations thereof.

In one aspect of this embodiment, the SSRI and HDACi are co-administered. In another aspect, the SSRI and HDACi are administered serially over time. In a further aspect, the SSRI and HDACi are part of a drug hybrid.

In the present invention, “co-administration” or “co-administering” means administration of two or more compounds together in the same composition, simultaneously in separate compositions, or as separate compositions administered at different times, as deemed most appropriate by a physician. As used herein, administered “serially” means administered at different times.

Another embodiment of the present invention is a method for identifying a patient population that suffers from a mood disorder that is more likely to respond to SSRI treatment. This method comprises:
(a) obtaining a biological sample from the patient;
(b) testing the biological sample to determine whether it has a reduced HDAC activity compared to a control population; and
(c) administering an effective amount of an SSRI to the patient, optionally together with an effective amount HDACi if the biological sample of the patient population has a reduced HDAC activity compared to the control population.

As used herein, a “biological sample” means a biological specimen. The biological sample may be a body fluid, a body tissue, or any portion thereof. Non-limiting examples of body fluids include whole blood, serum, plasma, interstitial fluid, saliva, ocular lens fluid, cerebro-spinal fluid, sweat, urine, stool, milk, ascites fluid, mucous, nasal fluid, sputum, synovial fluid, peritoneal fluid, vaginal fluid, menses, amniotic fluid, semen, gastric juice, vomit, lymph, and post-operative fluid collections. Non-limiting examples of body tissue include a biopsy and a scraping. The biopsy may be a bone or organ biopsy. The scraping may be a skin or mucosal scraping. Preferably, the biological sample is blood or peripheral blood lymphocytes.

Methods of measuring HDAC activity include, for example, measuring the gene expression levels of HDACs (including HDAC1, HDAC2, HDAC3, HDAC4, HDAC5, HDAC7, HDAC8, HDAC9, and HDAC10), or the levels of acetylated histone H4 proteins, as disclosed herein.

Suitable and preferred HDAC is and SSRIs are as set forth above.

Yet another embodiment of the present invention is a composition for treating or ameliorating the effects of a mood disorder. This composition comprises an effective amount of an SSRI, an HDACi, and a pharmaceutically acceptable carrier. Suitable HDACis and SSRIs are as set forth above.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions of the present invention may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject, e.g., patient, population. Accordingly, a given subject or subject, e.g., patient, population may fail to respond or respond inadequately to treatment.

As used herein, the terms “ameliorate”, “ameliorating” and grammatical variations thereof mean to decrease the severity of the symptoms of a disease in a patient.

The compositions according to the present invention may be in a unit dosage form comprising both SSRI and HDACi. In another aspect of this embodiment, the SSRI is in a first unit dosage form, and the HDACi is in a second unit dosage form, separate from the first.

A further embodiment of the present invention is a method for preventing, treating or ameliorating the effects of age-related cognitive deficits in a patient with a history of early life stress. This method comprises administering to the patient in need thereof an effective amount of an HDACi. In a preferred embodiment, the method further comprises administering to the patient an effective amount of an SSRI. Suitable HDACis and SSRIs are as set forth above.

As used herein, the terms “prevent”, “preventing” and grammatical variations thereof mean to administer a compound or a composition of the present invention to a patient who has not been diagnosed as having the disease or condition at the time of administration, but who could be expected to develop the disease or condition or be at increased risk for the disease or condition. Preventing also includes administration of at least one compound or a composition of the present invention to those subjects thought to be predisposed to the disease or condition due to age, familial history, due to the presence of one or more biological markers for the disease or condition and/or due to environmental factors.

As used herein, “age-related cognitive deficits” mean a decline in cognitive (thinking) function as a consequence of aging.

In one aspect of this embodiment, the age-related cognitive deficits are those governed by the medial prefrontal cortex (mPFC), such as, e.g., working memory and attention-set-shifting deficits, as well as hippocampal deficis, such as learning and memory.

An additional embodiment of the present invention is a composition for preventing, treating or ameliorating the effects of age-related cognitive deficits in a patient with a history of early life stress. This composition comprises an effective amount of an HDACi and a pharmaceutically acceptable carrier. In a preferred embodiment, the composition further includes an effective amount of an SSRI. Suitable HDACis and SSRIs are as set forth above.

Yet another embodiment of the present invention is a method for identifying and treating a patient population that suffers from a mood disorder that is more likely to respond to a combination SSRI/HDACi treatment. This method comprises:
(a) treating a patient population with an SSRI;
(b) determining which patients in the patient population are not responding to or who have low responsiveness to the SSRI treatment in step (a); and
(c) to those patients determined to be not responding to or to have low responsiveness to the SSRI treatment, administering the SSRI and an HDACi in an amount effective to treat the mood disorder.

As used herein, a “combination SSRI/HDACi treatment” means a treatment regimen in which an SSRI and an HDACi are co-administered.

In one aspect of this embodiment, the patient population has increased histone H4 protein acetylation levels. In another aspect of this embodiment, the patient population has reduced HDAC activity. Determination of whether a patient is not responding to an SSRI treatment or whether a patient has a low responsiveness to such a treatment is well known to those skilled in the art. Physicians and other appropriate medical professionals will make such determinations using accepted measures.

In the present invention, an “effective amount” or a “therapeutically effective amount” of a compound or composition disclosed herein is an amount of such compound or composition that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of mammal, e.g., human patient, and like factors well known in the arts of medicine and veterinary medicine. In general, a suitable dose of a composition according to the invention will be that amount of the composition, which is the lowest dose effective to produce the desired effect. The effective dose of a compound or composition of the present invention may be administered as two, three, four, five, six or more sub-doses, administered separately at appropriate intervals throughout the day. As noted above, the composition may be a drug hybrid or bifunctional drug containing at least one SSRI and at least one HDACi. This format is a preferred embodiment, particularly when the patient is an adolescent or a juvenile.

A suitable, non-limiting example of a dosage of a modulator of histone expression according to the present invention may be from about 1 ng/kg to about 1000 mg/kg. In general, however, doses employed for adult human treatment typically may be in the range of 0.0001 mg/kg/day to 0.0010 mg/kg/day, 0.0010 mg/kg/day to 0.010 mg/kg/day, 0.010 mg/kg/day to 0.10 mg/kg/day, 0.10 mg/kg/day to 1.0 mg/kg/day, 1.00 mg/kg/day to about 200 mg/kg/day. For example, the dosage may be about 1 mg/kg/day to about 100 mg/kg/day, such as, e.g., 2-10 mg/kg/day, 10-50 mg/kg/day, or 50-100 mg/kg/day. The dosage of a modulator of histone expression also may be about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, or 1000 mg/kg.

A suitable, non-limiting example of a dosage of the SSRI in the compositions disclosed herein is from about 0.1 to 100 mg/day, such as from about 0.5 mg/day to about 40 mg/day, including from about 1 mg/day to about 10 mg/day. Other representative dosages of such an agent include about 0.2 mg/day, 0.5 mg/day, 0.7 mg/day, 1 mg/day, 1.2 mg/day, 1.5 mg/day, 2 mg/day, 2.5 mg/day, 3 mg/day, 3.5 mg/day, 4 mg/day, 4.5 mg/day, 5 mg/day, 5.5 mg/day, 6 mg/day, 6.5 mg/day, 7 mg/day, 7.5 mg/day, 8 mg/day, 8.5 mg/day, 9 mg/day, 9.5 mg/day, 10 mg/day, 15 mg/day, 20 mg/day, 30 mg/day, 35 mg/day, 40 mg/day, 45 mg/day, 50 mg/day, 55 mg/day, 60 mg/day, 65 mg/day, 70 mg/day, 75 mg/day, 80 mg/day, 85 mg/day, 90 mg/day, 95 mg/day, and 100 mg/day.

As noted above, the effective dose of the SSRI or the modulator of histone expression in the compositions disclosed herein maybe administered as two, three, four, five, six or more sub-doses, administered separately at appropriate intervals throughout the day. In a preferred embodiment, particularly when the patient is an adolescent or juvenile, a bifunctional drug containing both an SSRI and an HDACi is administered once a day.

A composition of the present invention may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, a composition of the present invention may be administered in conjunction with other treatments. A composition of the present invention maybe encapsulated or otherwise protected against gastric or other secretions, if desired.

The compositions of the invention comprise one or more active ingredients in admixture with one or more pharmaceutically-acceptable carriers and, optionally, one or more other compounds, drugs, ingredients and/or materials. Regardless of the route of administration selected, the agents/compounds of the present invention are formulated into pharmaceutically-acceptable dosage forms by conventional methods known to those of skill in the art. See, e.g., Remington, The Science and Practice of Pharmacy (21^{st} Edition, Lippincott Williams and Wilkins, Philadelphia, Pa.).

Pharmaceutically acceptable carriers are well known in the art (see, e.g., Remington, The Science and Practice of Pharmacy (21^{st} Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier used in a pharmaceutical composition of the invention must be “acceptable” in the sense of being compatible with the other ingredients of the formulation and not injurious to the subject. Carriers suitable for a selected dosage form and intended route of administration are well known in the art, and acceptable carriers for a chosen dosage form and method of administration can be determined using ordinary skill in the art.

The compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Compositions of the present invention suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type maybe employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Compositions of the present invention for rectal or vaginal administration may be presented as a suppository, which maybe prepared by mixing one or more active ingredient(s) with one or more suitable nonirritating carriers which are solid at room temperature, but liquid at body temperature and, therefore, will melt in the rectum or vaginal cavity and release the active compound. Compositions of the present invention which are suitable for vaginal administration also include pessaries, tampons, creams, gels, pastes, foams or spray formulations containing such pharmaceutically-acceptable carriers as are known in the art to be appropriate.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable carrier. The ointments, pastes, creams and gels may contain excipients. Powders and sprays may contain excipients and propellants.

Compositions of the present invention suitable for parenteral administrations comprise one or more agent(s)/compound(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the active agent/drug then depends upon its rate of dissolution which, in turn, may depend upon crystal size and crystalline form. Alternatively, delayed absorption of a parenterally-administered agent/drug may be accomplished by dissolving or suspending the active agent/drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

The following examples are provided to further illustrate the methods of the present invention. These examples are illustrative only and are not intended to limit the scope of the invention in any way.

### EXAMPLES

### Example 1

### Materials and Methods

### Animals

Balb/cJ and C57BI/6J mice were housed in a temperature-controlled (26±2° C.) barrier facility with a 12-hour light/dark schedule (lights on at 6:00 A.M.) and had free access to food and water. All experiments involving animals were performed in accordance with the National Institutes of Health Guide for the Care and Use of Laboratory Animals (NIH Publication No. 80-23; revised 1996) and approved by the Institutional Animal Care and Use Committees at Columbia University.

### Infant Maternal Separation (IMS)

The IMS protocol was as previously described (Bhansali et al., 2007). Briefly, offspring of first-time mothers were separated from their dam daily for three hours (from 1:00 to 4:00 P.M.) from postnatal age day 2 (P2) until P15. Control animals were standard facility-reared (SFR) pups of first-time mothers. Housing and husbandry conditions were identical for IMS and SFR mice. Pups were weaned at P28 and group housed by sex (five animals randomly selected from at least five different litters). Since this study involved behavioral tests of emotive phenotypes that are sensitive to differences in the estrus cycle of females, all studies disclosed below were conducted on male mice.

### Drug Treatments

For chronic drug treatment, drugs were administered via the drinking water. Fluoxetine and theophylline (10⁻⁴ M) were dissolved in water, and mice consumed about 16 mg/kg and 32 mg/kg, respectively, per day. Suberoylanidide hydroxamic acid (SAHA) was dissolved in DMSO and subsequently diluted about 100 fold in water containing 0.2% sucrose. Mice consumed about 200 mg/kg/day of SAHA (Butler et al., 2000) and their controls received drinking water supplemented with the same amount of DMSO and sucrose that SAHA-treated animals received. Some mice were treated with both SAHA and fluoxetine. In these experiments, mice received a single intraperitoneal injection of SAHA (100 mg/kg) at P35, followed by SAHA treatment via the drinking water for the next 3 days. Then, fluoxetine (16 mg/kg/day) was added to the drinking water and the combined SAHA and fluoxetine treatment was continued until P60.

### Behavioral Tests

Mice were exposed to the Elevated Plus Maze (EPM) for 5 minutes as previously described (Mehta and Schmauss, 2011). Their times spent in open arms and the number of arm crossings was recorded. Three days later, mice were tested in a modified version of Forced Swim Test (FST), i.e., a 6 minute exposure on day 1 followed by another 6 minute exposure on day 2. On both days, the number of passive episodes and their duration (in seconds) were recorded during the last 4 minutes of FST exposure (see Bhansali et al., 2007), and the results obtained from the day 2 exposure were compared between the different treatment groups.

### Real-Time RT-PCR

Total RNA, extracted from freshly dissected neocortical tissue of the forebrain (whose caudal border is the mesodiencephalic junction), hippocampi, and striatae via guanidine/cesium chloride ultracentrifugation, served as a template for first-strand cDNA synthesis using Murine Moloney Leukemia Virus reverse transcriptase (USB, Cleveland, Ohio). Real time PCR was performed using the iQ Real Time PCR detection System (Bio-Rad, Hercules, Calif.) and SYBR Green (Bio-Rad). The primers for amplification of HDAC mRNAs, shown in Table 1, were designed such that they fit a single PCR protocol with a transcript length of 50-100 base pairs. Cycle thresholds (Ct) of amplification (normalized to β-actin whose Ct values did not differ between groups) were expressed as ½^{ΔCt} values, i.e., higher numbers reflect higher expression.

TABLE 1
List of primers used for real-time quantitative RT-PCR^{a}

| mRNA | Accession # | Forward primer | Reverse primer |
|---|---|---|---|
| HDAC1 | NM_008228 | CACGCCAAGTGTGTGGAGTT | CATCAGCATGGGCAAGTTGA |
| | | (SEQ ID NO: 1) | (SEQ ID NO: 2) |
| HDAC2 | NM_008229 | GCTGCGACTGGCTTCAACTAC | GCTGCTTGGCTTCACTAGGC |
| | | (SEQ ID NO: 3) | (SEQ ID NO: 4) |
| HDAC3 | NM_010411 | GTGATCGATTAGGCTGCT | ATTCCCCATGTCCTCGAATG |
| | | (SEQ ID NO: 5) | (SEQ ID NO: 6) |
| HDAC4 | NM_207225 | CCTCGAGAATGTGATCAGGGA | GGCCTTGACGTTTGAGAGCA |
| | | (SEQ ID NO: 7) | (SEQ ID NO: 8) |
| HDAC5 | NM_010412 | GGAGGACTGCATTCAGGTCAA | TCATCAGGACCACTCTCGCC |
| | | (SEQ ID NO: 9) | (SEQ ID NO: 10) |
| HDAC7 | NM_019572 | CTGGGCAGGTAGTCAGGTCC | TTGGGATAGCCGTCAGGGT |
| | | (SEQ ID NO: 11) | (SEQ ID NO: 12) |
| HDAC8 | NM_027382 | GTGCCTGATTGACGGGAAGT | CCACCCTCCAGACCAGTTGAT |
| | | (SEQ ID NO: 13) | (SEQ ID NO: 14) |
| HDAC9 | NM_024124 | TCAGCTGAGAGCAGGCTGTG | TCAGAAGGGCTGACGGTTG |
| | | (SEQ ID NO: 15) | (SEQ ID NO: 16) |
| HDAC10 | NM_199198 | TGGAGGGTTTCTGAGCCTCA | CCATAGGCCAAGGGCAGTAC |
| | | (SEQ ID NO: 17) | (SEQ ID NO: 18) |

^{a}The nucleotide sequences for the targeted mRNA sequences were derived from the NCBI Data Base using the accession numbers listed above.

### Western Blotting

Immunoblotting was performed as described previously (Levine et al., 2005) with some modifications. Forebrain neocortical tissue was dissected in phosphate-buffered saline supplemented with protease inhibitors and homogenized in 0.5% SDS, protease inhibitor cocktail (Complete Mini, Roche, Germany), 10 mM EDTA, and 10 mM Tris HCl (pH 8.0). Protein concentration was measured using the NanoDrop instrument (Thermo Scientific, Wilmington, Del.). For each sample, fifty micrograms of protein were separated on 15% SDS/PAGE gels, transferred to nitrocellulose membranes (BioRad) and incubated overnight at 4° C. with one of the following rabbit polyclonal antibodies from Millipore (Billerica, Mass.): anti-acetyl histone H4K5 (1:1,000,000), anti-acetyl histone H4K8 (1:100,000), anti-acetyl histone H4K12 (1:20,000), anti-acetyl histone H4K16 (1:100,000), pan histone H4 (1:10,000), pan histone H3 (1:500,000), antidimethyl-histone H3 (Lys9) (1:1,000), and anti-trimethyl-histone H3 (Lys4) (1:100,000).

For loading controls, an anti-GAPDH antibody was used (Abcam, Cambridge, Mass.; 1:100,000). After incubation with primary antibody, membranes were incubated with a horseradish-peroxidase-conjugated secondary antibody (anti-rabbit IgG-HRP; Sigma, St. Louis, Mo.; dilution: 1:5,000) for 1 hour at room temperature. SuperSignal West Dura (Thermo Scientific, Waltham, Mass.) was used to visualize bound antigen. Optical densities (OD) of histone protein signals were determined using NIH ImageJ software. ODs were normalized to corresponding ODs obtained for GAPDH.

### Statistical Analysis

For comparisons between two groups of animals (SFR and IMS), two-tailed Student's t tests were used. For experiments involving multiple groups, one-way analysis of variance (ANOVA) (effect of postnatal age or treatment) was used, and statistical differences were resolved post hoc using Tukey-Kramer Multiple Comparisons tests. All statistical analyses were carried out using Graph Pad InStat Version 3.0 (GraphPad Software, San Diego, Calif.).

### Example 2

### Biphasic Changes in HDAC mRNA Expression in the Forebrain Neocortex of Balb/c Mice Exposed to Early Life Stress

Real-time PCR was used to compare the forebrain neocortical expression levels of mRNA encoding class I and class II HDACs (de Ruitjer et al., 2003) between IMS Balb/c and their SFR controls during postnatal development. At P15 (the end of IMS), none of the HDACs mRNA expression levels examined differed between IMS Balb/c mice and their SFR controls. For the individual HDACs, the ½^{ΔCt} values determined in real-time PCR experiments for SFR controls and IMS mice, respectively, were as follows: HDAC 1 (means±sem): 0.007±0.002 and 0.008±0.0005; HDAC2: 0.0015±0.0004 and 0.0011±0.0001; HDAC3: 0.043±0.01 and 0.038±0.0016; HDAC4: 0.015±0.004 and 0.014±0.001; HDAC5: 0.028±0.004 and 0.028±0.006; HDAC7: 0.0033±0.001 and 0.0037±0.001; HDAC8: 0.0027±0.001 and 0.0020±0.0001; HDAC9: 0.0027±0.0008 and 0.0026±0.0004; HDAC10: 0.0038±0.001 and 0.0049±0.001).

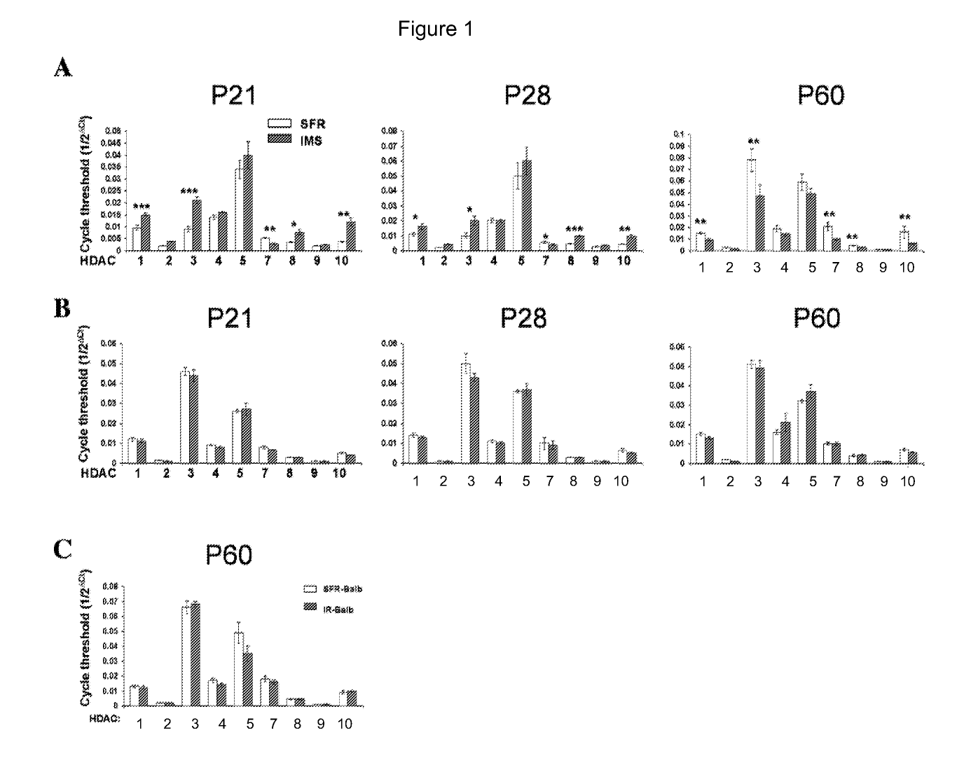

However, significant differences in HDAC mRNA expression emerged at P21 (early adolescence): While the expression of HDACs 2, 4, 5, and 9 did not differ between SFR controls and IMS Balb/c mice, HDACs 1, 3, 8 and 10 were expressed at significantly higher levels in IMS mice relative to SFR controls, an effect that was still detected at P28 (FIG. 1A). In addition, HDAC7 mRNA expression was also affected, but its expression was significantly lower in IMS Balb/c mice at these postnatal ages (FIG. 1A).

A different result was obtained at later postnatal ages. At P35 (the end of early adolescence), the expression levels of several HDAC mRNAs became lower in IMS mice compared with SFR controls. Specifically, two-tailed Student's t tests revealed lower expression of HDAC1 (p<0.01), HDAC3 (p<0.03), HDAC7 (p<0.03) and HDAC8 (p<0.03) and also decreased expression of HDAC 10 that did not reach significance (p=0.096). This change in HDAC mRNA expression persisted into adulthood. As shown in FIG. 1A, P60 IMS Balb/c mice exhibited significantly lower mRNA expression of HDACs 1, 3, 7, 8, and 10 than SFR controls, but, like at P21, the expression of HDACs 2, 4, 5, and 9 was unaltered.

Interestingly, for SFR controls, a comparison of the mRNA expression levels of all 9 HDACs at defined postnatal ages (P15, P21, P28, P35, and P60) by one-way ANOVA revealed significant developmental changes in expression of the 5 HDACs affected by early life stress (HDAC1: F(4,22)=6.712, p=0.0011; HDAC3: F(4,22)=8.94, p=0.0002; HDAC7: F(4,22)=6.306, p=0.0015; HDAC8: F(4,22)=8.34, p=0.0003; HDAC10: F(4,22)=6.305, p=0.0015). Specifically, HDAC1 and HDAC8 mRNA levels increased gradually after P15 to reach mature levels at P35 (mid-adolescence). HDAC3 mRNA was expressed at the highest levels at P15 and P60, but exhibited significantly lower expression between P21 and P35 (early to mid-adolescence). HDAC7 and HDAC10 reached their highest expression levels only by P60. In contrast, while all HDACs affected by IMS exposure undergo expression changes during normal postnatal development, the majority of the unaffected HDACs do not. One exception is HDAC5 mRNA (ANOVA, F(4,22)=9.574, p=0.0001) which, like HDACs 7 and 10, reached highest expression levels at P60.

In IMS Balb/c mice, altered HDAC mRNA expression did not occur throughout the brain. In contrast to the expression changes in the forebrain neocortex (FIG. 1A), none of the nine HDACs examined had altered expression in the hippocampus, neither between P21 and P28 nor at P60 (FIG. 1B), and the same was found for the striatum (not shown). Moreover, even in the forebrain neocortex, HDAC mRNA expression was unaffected in Balb/c mice that were exposed to a powerful adolescent stressor, namely isolation rearing from P28 to P60 (FIG. 1C) indicating that the timing of stress exposure during postnatal development (i.e., infancy versus adolescence) is a critical determinant of the effect of stress on forebrain neocortical HDAC mRNA expression.

### Example 3

### Altered Histone Modifications in IMS Balb/c Mice

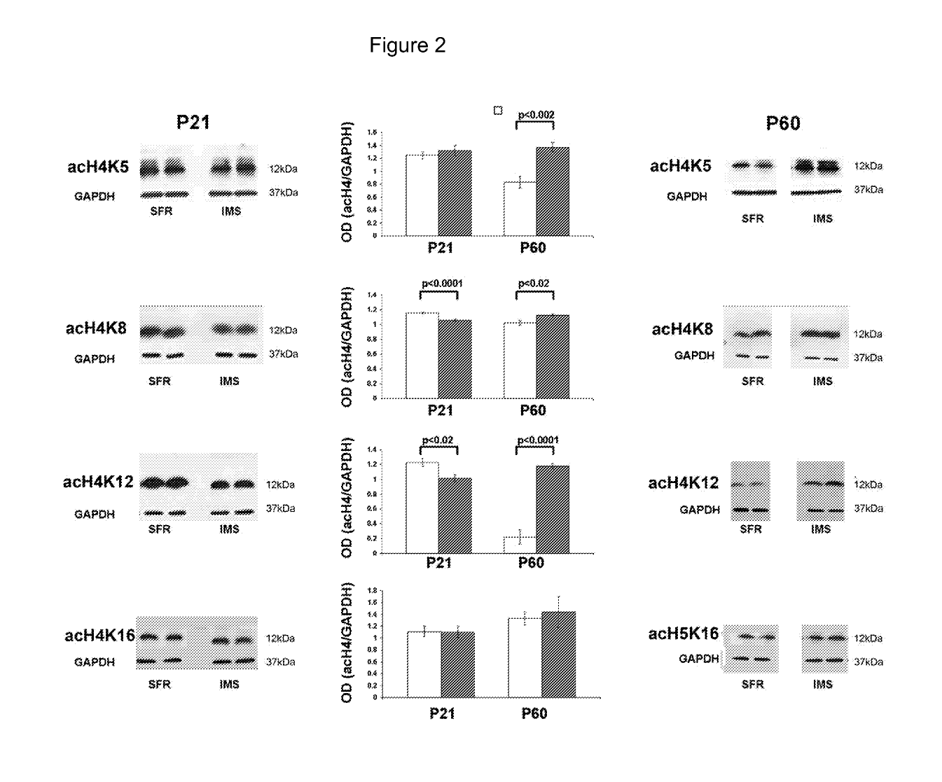

If changes in HDAC mRNA expression lead to changes in HDAC activity that is functionally relevant, changes in histone acetylation must occur. To test this, Western blots were used to measure the expression of acetylated histone H4 and H3 protein in the forebrain neocortex. As expected, no differences were found between SFR controls and IMS Balb/c mice at P15 (not shown), but altered histone modifications became evident at P21. As shown in FIG. 2, like the biphasic changes in HDAC mRNA expression in IMS Balb/c mice, the expression of histone H4 protein acetylated at lysine (K) position 8 (K8) and K12 was also altered in the biphasic manner. Expression significantly dropped at P21 and significantly increased at P60 relative to SFR controls. Moreover, at P60, H4 protein acetylated at K5 was also increased, but the expression of H4 protein acetylated at K16 was unaffected (FIG. 2).

The changes in histone H4 acetylation in IMS Balb/c mice occurred in the absence of changes in total H4 protein expression, and neither the expression of total histone H3 protein nor the expression of H3 protein acetylated at K9 were altered in these 13 mice (Table 2). In addition, because histone H3 methylation could facilitate H4 acetylation (Wang et al., 2009), expression levels of di- and tri-methylated H3 protein at P21 and P60 were also measured. As shown in Table 2, there was no change in either form of methylated H3 protein expression at P21, and at P60, the trimethylated H3 protein expression was also not significantly altered in IMS mice. Only the expression of dimethylated H3 was increased in P60 IMS Balb/c mice.

TABLE 2
Expression of total histone H3 and H4 protein and acetylated
or methylated H3 protein in the forebrain neocortex of SFR and
IMS Balb/c mice at postnatal ages P21 and P60^{a}

| | P21 | | P60 | |
|---|---|---|---|---|
| | SFR | IMS | SFR | IMS |
| H4 | 0.92 ± 0.03 | 0.80 ± 0.06 | 0.78 ± 0.09 | 0.83 ± 0.05 |
| H3 | 1.50 ± 0.11 | 1.74 ± 0.11 | 0.90 ± 0.13 | 0.83 ± 0.06 |
| acH3K9 | 0.56 ± 0.06 | 0.67 ± 0.02 | 0.79 ± 0.02 | 0.76 ± 0.02 |
| H3K9me2 | 0.88 ± 0.03 | 0.89 ± 0.03 | 0.75 ± 0.02 | 0.88 ± 0.01* |
| H3K4me3 | 1.57 ± 0.09 | 1.59 ± 0.10 | 1.11 ± 0.03 | 1.19 ± 0.05 |

^{a}Data are mean ± sem of optical densities on Western blots (n = 6 per group) that were normalized to GAPDH optical densities and compared by two-tailed Student's t test.
*p < 0.002 compared to SFR P60 mice.

Thus, in the forebrain neocortex of IMS Balb/c mice, changes in histone H4 acetylation follow the biphasic developmental changes observed for the expression of HDAC mRNA. This was most evident for the expression of acetylated histone H4K12 protein. In contrast to the biphasic changes in histone H4 acetylation, increased dimethylation of H3 protein was only observed at P60.

In sum, when inbred strains of mice were exposed to a prominent risk factor for mood disorders, namely early life stress (ELS), the stress-susceptible strain Balb/c exhibits persistent changes in gene expression and emotive behavior after ELS exposure. Such changes in gene expression involve decreased expression of class I histone deacetylases (HDACs) and increased expression of acetylated histone H4 protein in the forebrain neocortex (summarized in Table 3 below).

TABLE 3
Changes in Histone Modifications in the Forebrain Neocortex of IMS
Balb/c Mice at P60^{a}

| | HDAC mRNA | H4 | acH4K12 | acK4K8 | acH4K5 | acH4K16 | H3 | acH3K9 | H3K9me2 | H3K4me3 |
|---|---|---|---|---|---|---|---|---|---|---|
| P60 | Decreased: | = | Increased | Increased | Increased | = | = | = | Increased | = |
| | HDACs1, 3, 8, and | | p < 0.001 | p < 0.008 | p < 0.008 | | | | p < 0.02 | |
| | 10 | | | | | | | | | |

^{a}Data (n = 5/group) were compared to standard-facility-reared Balb/c controls (two-tailed t tests);
=: unaltered expression.

### Example 4

### Unaltered HDAC Expression and Histone Modifications in the Forebrain Neocortex of IMS C57BI/6 Mice

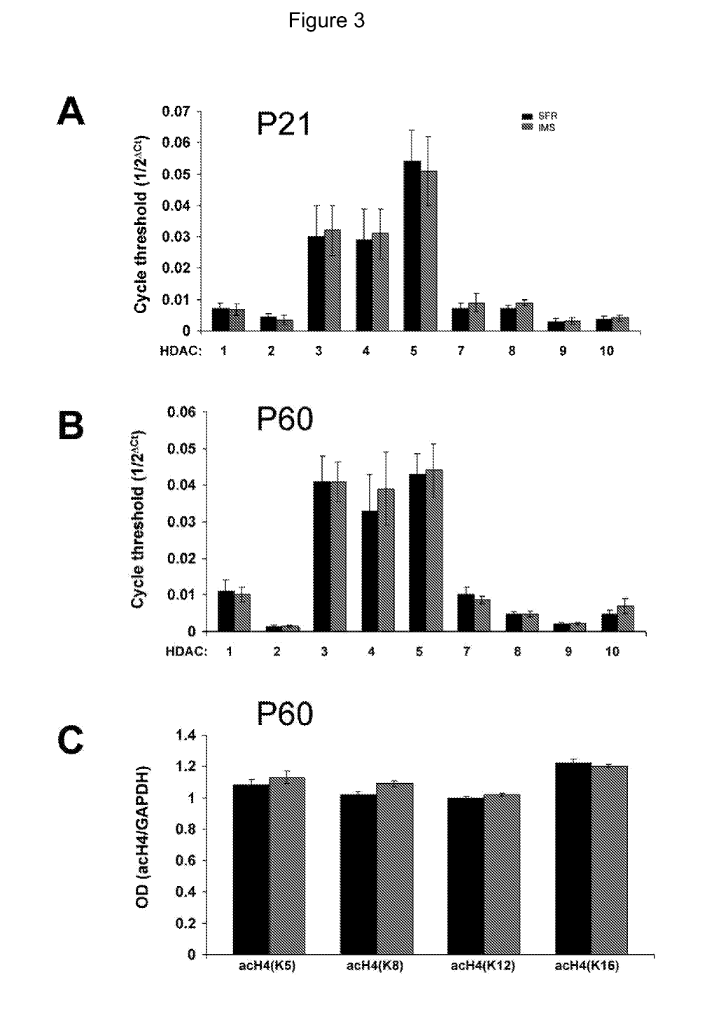

In contrast to the biphasic changes in HDAC expression detected in P21 and P60 IMS Balb/c mice, HDAC mRNA expression was unaltered in IMS C57BI/6 mice at these ages (FIGS. 3A and 3B). Moreover, none of the changes in histone modifications detected in IMS Balb/c mice were detected in C57BI/6 mice (FIG. 3C, Table 4). Thus, HDAC-triggered changes in post-translational histone modifications are strain-specific and, interestingly, occur in the stress-susceptible strain and not in the resilient strain.

TABLE 4
Expression of total histone H3 and H4 protein and
acetylated or methylated H3 protein in the forebrain
neocortex of SFR and IMS C57BI/6 mice at P60^{a}

| | IMS | IMS-fluoxetine |
|---|---|---|
| H4 | 0.98 ± 0.02 | 1.00 ± 0.03 |
| H3 | 0.97 ± 0.02 | 0.98 ± 0.02 |
| acH3K9 | 1.00 ± 0.02 | 1.00 ± 0.03 |
| H3K9me2 | 0.85 ± 0.02 | 0.89 ± 0.03 |
| H3K4me3 | 0.90 ± 0.032 | 0.94 ± 0.02 |

^{a}Data are mean ± sem of optical densities on Western blots (n = 5 per group) that were normalized to GAPDH optical densities. Two-tailed Student's t tests revealed no significant differences between groups.

There are two notable differences between SFR control mice of both strains at P60: One is that, although the majority of HDAC mRNAs were expressed at equal levels, HDAC3 mRNA was roughly twice as abundant in Balb/c mice. In P60 IMS Balb/c mice, however, the reduction of HDAC3 mRNA expression led to mRNA levels that were similar to those found in SFR C57BI/6 mice (FIGS. 1 and 3). In addition, while the majority of the histone variants were expressed at equal levels in SFR controls of both strains, there was a difference in the levels of acetylated H4K12 protein. Its expression was 3-fold lower in Balb/c mice and, strikingly, only the increased expression of acetylated H4K12 in IMS Balb/c mice led to expression levels comparable to that of C57BI/6 mice. Thus, in IMS Balb/c mice, changes in expression of two epigenetic modulators (HDAC3 and acetylated H4K12) abolished the differences in expression normally found between SFR controls of both strains. These data suggest that the resilience of C57BI/6 mice to early life stress exposure could, at least in part, be due to their lower levels of HDAC3 expression and their higher levels of acetylated H4K12.

### Example 5

### Chronic Activation of HDACs in Adolescent IMS Balb/c Mice Decreases the Adult Histone H4K12 Hyperacetylation Phenotype

To test whether the increased histone H4 acetylation found in IMS Balb/c mice can be reversed by stimulating HDACs during mid- to late adolescence, IMS Balb/c mice were chronically treated with theophylline (10⁻⁴M in drinking water) from P35 to P60. Thus, over 24 hours of theophylline-supplemented drinking water consumption, the plasma concentrations of theophylline are below 10⁻⁴M, i.e., a concentration at which theophylline activates HDACs (specifically HDAC1 and HDAC3, but not HDAC2) but exerts no antagonist effect on adenosine receptors or inhibition of phosphodiesterases (Ito et al., 2002).

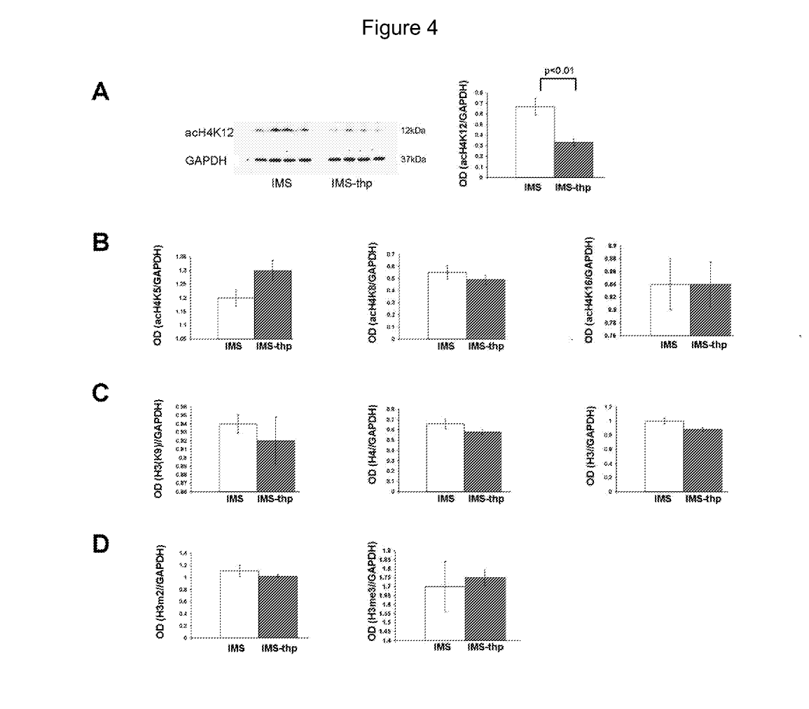

In IMS Balb/c mice, this theophylline treatment did not alter the expression levels of any of the nine HDAC mRNAs studied here (not shown). However, it significantly reduced expression of acetylated H4K12 protein compared to non-treated IMS controls (FIG. 4). The expression of acetylated histone H4K5, H4K8, H4K16 (FIG. 4B), acetylated histone H3K9 and total H3 and H4 protein (FIG. 4C), and the expression of di- and trimethylated H3 protein remained unaltered (FIG. 4D). Thus, theophylline reversed the most prevalent histone modification resulting from early life stress exposure, namely increased acetylation of histone H4K12.

### Example 6

### The Emotional Phenotype of Theophylline-Treated IMS Balb/c Mice

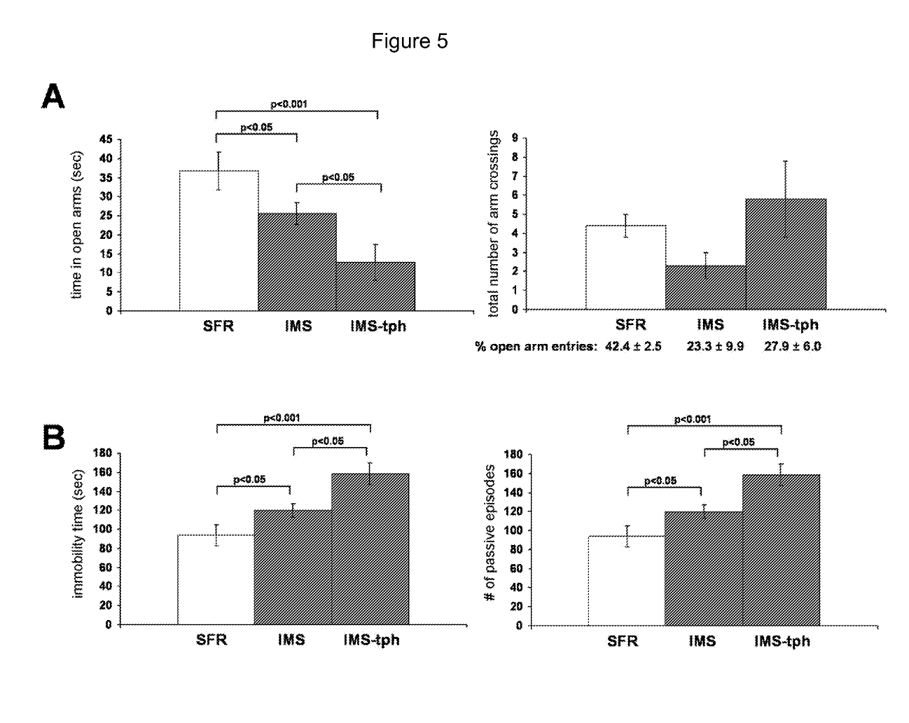

Because the most prominent IMS-induced histone modification is reversible by adolescent theophylline treatment, whether the hyperacetylated histone H4K12 phenotype is an adaptive or mal-adaptive epigenetic process in terms of the behavioral phenotype was investigated. Furthermore, because the inventors have previously shown that adult IMS Balb/c mice exhibit increased anxiety-like behavior in the Elevated Plus Maze (EPM) test as well as depression-like behavior in the Forced Swim test (FST) (Bhansali et al., 2007, Mehta and Schmauss, 2011), whether IMS Balb/c mice treated with theophylline during mid- to late adolescence (P35 to P60) exhibit differences in these behavioral phenotypes when compared to non-treated IMS mice was tested. A one-way ANOVA revealed significant differences in the total time spent in open arms of the EPM between SFR controls, IMS mice, and theophylline-treated IMS Balb/c mice (F(2,17)=7.288, p=0.0052). Consistent with the previous findings, IMS Balb/c mice spent significantly less time in the open arms compared with SFR controls (FIG. 5A). Strikingly, theophylline-treated IMS Balb/c mice exhibited a more severe anxiety-like phenotype, i.e., they spent significantly less time in the open arms than non-treated IMS mice (FIG. 5A). There was no significant difference in the total number of arm crossings between the three group of mice (ANOVA, F(2,20)=1.74, p=0.20) indicating that the difference in times spend in open arms is not due to decreased locomotor activity of non-treated and theophylline-treated IMS Balb/c mice (FIG. 5A). Moreover, although non-treated and theophylline-treated IMS Balb/c mice exhibited lower percentages of crossings into open arms compared with SFR controls, this difference did not reach statistical significance (ANOVA, F(3,20)=3.28; p=0.06) (FIG. 5A). Thus, the main difference between the groups of mice is the total time spent in open arms.

A similar result was obtained in the FST. Although there were no significant differences in the number of passive episodes between SFR controls, IMS mice, and theophylline-treated IMS mice, ANOVA revealed significant differences in the total immobility time (i.e., time spent passively floating) between the three groups of mice F(2,16)=10.26, p=0.0014). As shown in FIG. 5B, IMS Balb/c mice exhibited greater immobility compared with SFR controls, a phenotype that was also significantly potentiated after theophylline treatment.

These data illustrate that the theophylline-induced decrease in acetylated H4K12 expression in IMS Balb/c mice worsened their emotional behavioral phenotype, suggesting that the H4K12 hyperacetylation triggers distinct changes in gene expression that ameliorate the severity of the emotive phenotype resulting from early life stress.

### Example 7

### The Effect of Adolescent Fluoxetine Treatment on Histone Modifications in IMS Balb/c Mice

The inventor has previously shown that adolescent treatment with the antidepressant drug fluoxetine (a selective serotonin re-uptake inhibitor) effectively reversed the abnormal behavior of IMS Balb/c mice in the FST (Bhansali et al., 2007). Because this is in contrast to the effects of theophylline treatment shown above, whether adolescent treatment with fluoxetine affects either HDAC mRNA expression and/or histone modifications in IMS Balb/c mice in a manner opposite to the theophylline-treatment paradigm was investigated. Indeed, fluoxetine treatment did not alter HDAC mRNA expression at P60 (real-time PCR cycle thresholds (½^{ΔCt} (IMS/IMS-fluoxetine, mean±sem)): HDAC1: 0.0094±0.0001/0.011±0.0006, HDAC2: 0.0017±0.0001/0.0016±0.0001, HDAC3: 0.047±0.006/0.055±0.004, HDAC4: 0.014±0.001/0.011±0.002, HDAC5: 0.049±0.004/0.044±0.006, HDAC7: 0.011±0.001/0.011±0.001, HDAC8: 0.0032±0.0001/0.0035±0.0004, HDAC9: 0.001±0.0002/0.001±0.0002, HDAC10: 0.0065±0.0008/0.0074±0.0006). Fluoxetine treatment, however, triggered changes in histone modifications in the forebrain neocortex of IMS Balb/c that were strikingly different. Fluoxetine increased total histone H3 and H4 protein expression and significantly increased the amounts of acetylated histones H3K9, H4K8, and H4K12 compared to non-treated IMS mice (Table 5). Thus, in contrast to the effects of adolescent theophylline treatment, adolescent fluoxetine treatment globally augmented histone H3 and H4 expression and further elevated the expression of the acetylated histone H4 proteins that were already increased after early life stress exposure alone. While the fluoxetine-induced increase in total histone H3 and H4 protein expression was 60% and 13%, respectively, the increase in acetylated H3K9 and H4K12 proteins was even larger (55% and 230%, respectively). Thus, fluoxetine treatment also altered the ratio of acetylated to non-acetylated protein in favor of increased histone acetylation. Finally, adolescent fluoxetine also increased the expression of trimethylated histone H3 protein, but did not change the expression of dimethylated histone H3 protein (Table 5) that was elevated in IMS Balb/c mice (Table 2).

TABLE 5
Expression of total and post-translationally modified histone
H3 and H4 proteins in the forebrain neocortex of Balb/c mice treated
with fluoxetine during adolescence^{a}.

| | IMS | IMS-fluoxetine |
|---|---|---|
| H4 | 0.62 ± 0.05 | 0.97 ± 0.05** |
| acH4K5 | 0.92 ± 0.01 | 0.94 ± 0.02 |
| acH4K8 | 1.10 ± 0.02 | 1.51 ± 0.06* |
| acH4K12 | 0.50 ± 0.11 | 1.16 ± 0.04*** |
| acH4K16 | 0.96 ± 0.03 | 1.04 ± 0.04 |
| H3 | 1.16 ± 0.01 | 1.31 ± 0.03** |
| acH3K9 | 0.78 ± 0.03 | 1.21 ± 0.02*** |
| H3K9me2 | 1.00 ± 0.08 | 0.93 ± 0.03 |
| H3K4me3 | 0.61 ± 0.03 | 0.94 ± 0.07* |

^{a}Data are mean ± sem of optical densities on Western blots (n = 5 per group) that were normalized to GAPDH optical densities and compared by two-tailed Student's t test.
*p < 0.02;
**p < 0.001;
***p < 0.0007.

### Example 8

### Inhibiting HDAC Activity Increases the Antidepressant Effects of Fluoxetine

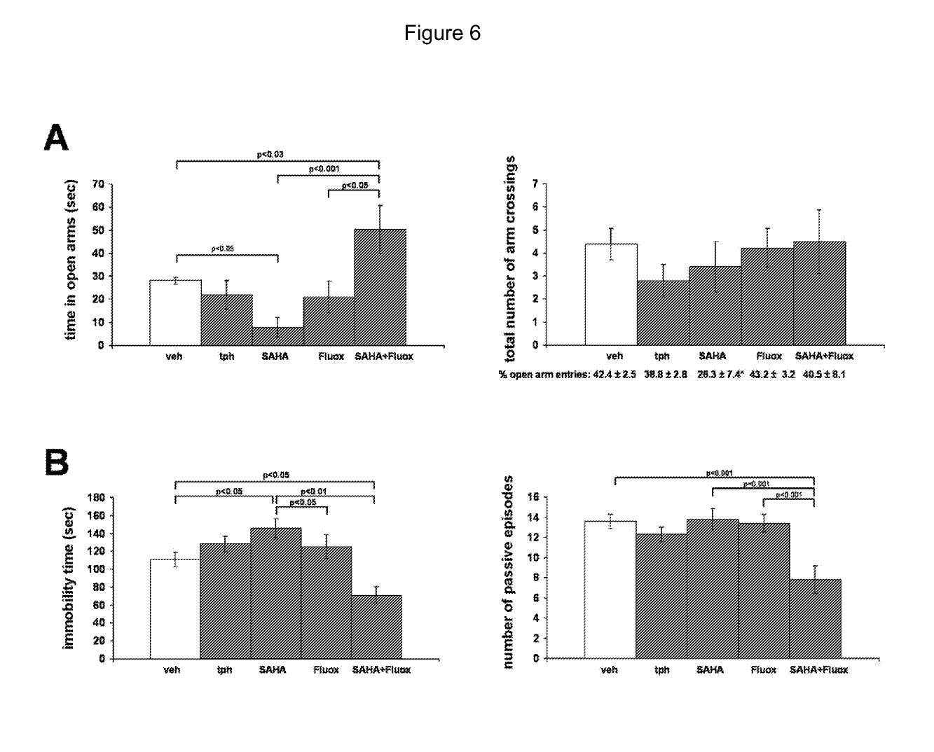

In contrast to the potent antidepressant effects of adolescent fluoxetine treatment detected in IMS Balb/c mice, the same treatment had no effect on the FST behavior of SFR controls (Bhansali et al., 2007). Because the data from IMS Balb/c mice suggest that their reduced HDAC activity and the resultant histone modifications enhance the antidepressant efficacy of fluoxetine, whether co-treating SFR mice with an HDAC inhibitor and fluoxetine would also enhance the effects of fluoxetine was investigated. Thus, SFR mice were treated with the class I/II HDAC inhibitor SAHA (200 mg/kg/day) between P35 and P60 (a treatment known to increase both H3 and H4 acetylation (Butler et al., 2000; Hockly et al., 2003)) either alone or in combination with fluoxetine. The effects of these treatments on EPM and FST behavior were first measured. For comparison, SFR mice treated only with theophylline or fluoxetine during adolescence were also included. Although a one-way ANOVA revealed no significant differences for the total number of arm crossings between non-treated SFR controls and the 4 groups of treated mice (F(4,37)=1.062; p=0.39), there were significant differences in the times spent in the open arms of the EPM (F(4,29)=6.934; p=0.0005). While SFR mice treated only with theophylline or fluoxetine did not differ from non-treated controls and, while SFR Balb/c mice treated only with SAHA spent significantly less time in the open arms, SFR mice treated with SAHA and fluoxetine spent significantly more time in the open arms (FIG. 6A). Moreover, ANOVA revealed no significant differences in the percentages of open arm entries between the different treatment groups (F(2,37)=1.835; p=0.15). It is, however, noted that SAHA-treated SFR mice did exhibit the lowest percentage of open arm entries (see FIG. 6A). Nevertheless, similar to the results shown in FIG. 5, the main difference between the treatment groups resides in the total time spent in open arms.

A similar result was obtained for the FST. ANOVA revealed significant differences between the five treatment groups for both the total time spent in immobility (F(4,42)=3.91; p=0.0087) and the number of passive episodes (F(4,42)=11.481, p<0.0001). Similar to the results obtained from the EPM test, SFR mice treated only with theophylline or fluoxetine did not differ from SFR controls, and SFR Balb/c mice treated only with SAHA exhibited significantly increased immobility in the FST (FIG. 6B). However, compared with all other groups, mice treated with SAHA and fluoxetine exhibited not only significantly decreased immobility but also a significantly reduced number of passive episodes (FIG. 6B). These data illustrate that HDAC inhibition also enhances the antidepressant efficacy of adolescent fluoxetine treatment in non-stressed Balb/c mice.

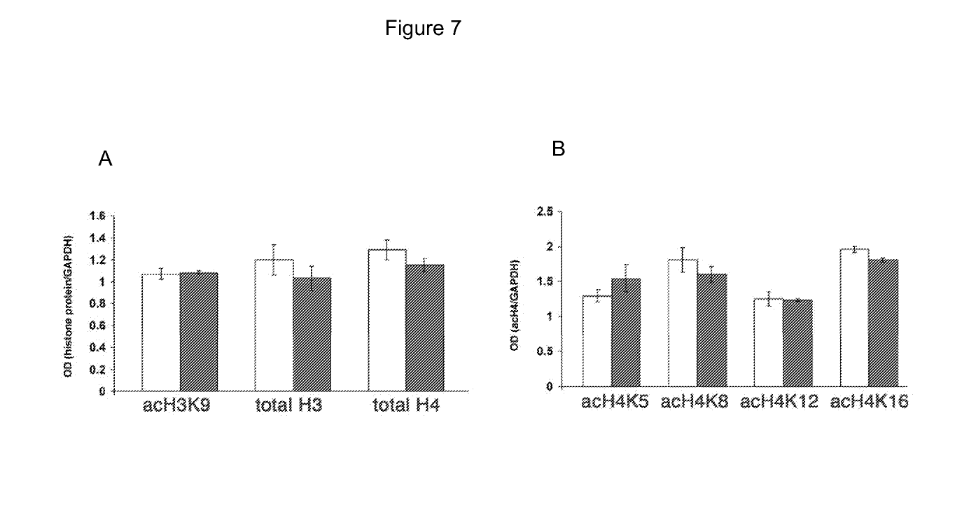

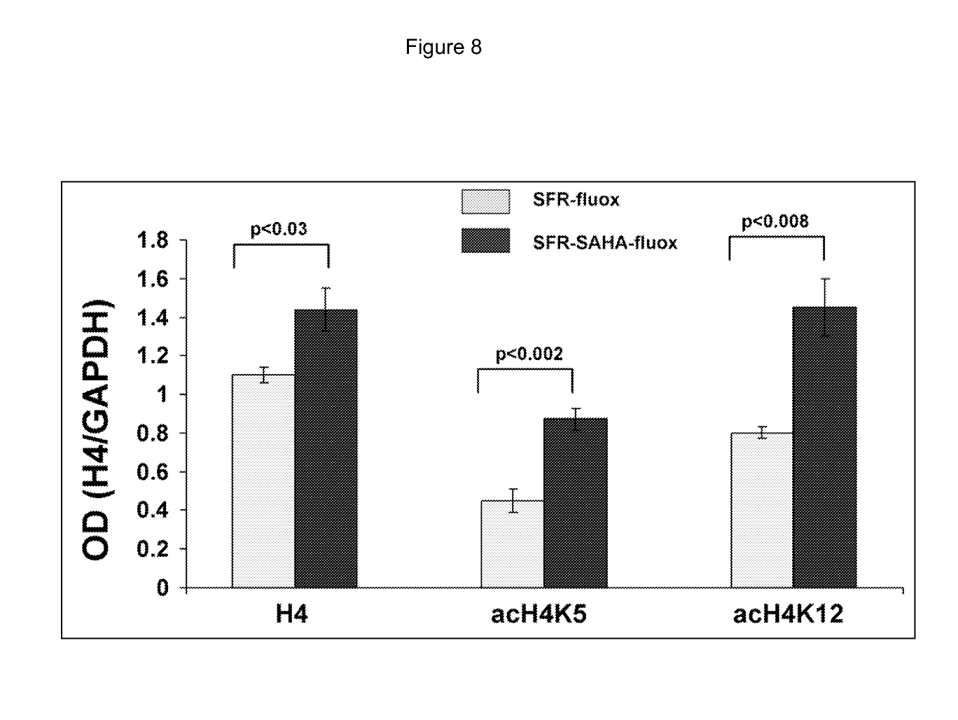

In sum, the ELS-triggered epigenetic phenotype was introduced in control mice by treating them chronically with the HDAC inhibitor SAHA during adolescence (FIG. 8). For comparison, a group of mice that were treated with the HDAC activator theophylline (tph) during adolescence was also included. Then, the effect of adolescent fluoxetine in SAHA-treated and non-treated mice was compared. As shown in FIG. 6, only co-treatment with SAHA and fluoxetine, but not fluoxetine treatment alone, led to improved emotive behavior in the Elevated Plus Maze (EPM) and the Forced Swim Test (FST). SAHA/fluoxetine co-treatment also increased expression of total H4 and acetylated histone H4K5 (aCH4K5) and aCH4K12 proteins (FIG. 8), i.e., an effect not detected in SFR mice treated only with fluoxetine (FIG. 7). Thus, the effect of fluoxetine on histone H4 expression in SAHA-co-treated SFR mice is very similar to the effect of fluoxetine alone in IMS mice with naturally reduced HDAC activity.

### Example 9

### Increased Expression of Histone H4 Protein in SFR Mice Treated with SAHA and Fluoxetine During Adolescence

Because of the enhanced behavioral responsiveness of SAHA-treated SFR mice to fluoxetine treatment, whether the SAHA-induced decrease in HDAC activity in SFR mice also affected the expression of histone H3 and H4 proteins in response to fluoxetine treatment, i.e., an effect detected in fluoxetine-treated IMS mice with naturally occurring reduced HDAC activity was tested. As shown in Table 6, in SAHA-treated SFR mice, fluoxetine also altered histone modifications, and there is substantial overlap between the fluoxetine-induced changes in SAHA-treated SFR mice and fluoxetine treated IMS mice. Compared to SFR mice treated only with fluoxetine, mice co-treated with SAHA exhibited significantly increased total histone H4 expression along with increased expression of acetylated H4K5 and H4K12 proteins. Also the expression of acetylated histone H4K8 was increased, but only a trend towards significance (p=0.07) was found for this protein. Thus, while there are subtle differences, in general, fluoxetine affected histone H4 expression similarly in SAHA-treated SFR mice and in IMS mice (Tables 5 and 6) and, also in SAHA-treated SFR mice, the increase in acetylated H4K5 and H4K12 expression (93% and 81%, respectively) was larger than the increase in total H4 expression (31%). In contrast to fluoxetine-treated IMS mice, however, in SAHA-treated SFR mice, fluoxetine did not affect the expression of total H3 and acetylated H3K9 proteins. However, like in fluoxetine treated IMS mice, SAHA-treated SFR mice also exhibited increased expression of trimethylated histone H3K4 protein (Table 6). Thus, these data suggest that increased expression of acetylated histone H4 and trimethylated histone H3 play a functional role in mediating the behavioral responsiveness to adolescent fluoxetine treatment.

TABLE 6
Expression of total and post-translationally modified histone
H3 and H4 proteins in the forebrain neocortex of SFR Balb/c mice
treated with fluoxetine alone or in combination with SAHA^{a}.

| | SFR-fluox | SFR-SAHA-fluox |
|---|---|---|
| H4 | 1.10 ± 0.04 | 1.44 ± 0.11* |
| acH4K5 | 0.45 ± 0.06 | 0.87 ± 0.06*** |
| acH4K8 | 0.86 ± 0.06 | 1.06 ± 0.08 |
| acH4K12 | 0.80 ± 0.03 | 1.45 ± 0.15** |
| acH4K16 | 0.70 ± 0.06 | 0.71 ± 0.09 |
| H3 | 0.96 ± 0.01 | 0.97 ± 0.01 |
| acH3K9 | 0.86 ± 0.03 | 1.15 ± 0.18 |
| H3K9me2 | 1.08 ± 0.04 | 1.07 ± 0.03 |
| H3K4me3 | 1.12 ± 0.02 | 1.33 ± 0.03*** |

^{a}Data are mean ± sem of optical densities on Western blots (n = 5 per group) that were normalized to GAPDH optical densities and compared by two-tailed Student's t test.
*p < 0.03;
**p < 0.008;
***p < 0.002.

Studies on mice exposed to early life stress and raised to adulthood without further stress exposure can provide valuable insight into the development of adaptive and maladaptive processes that ultimately shape the adult phenotype. It was found that, in the stress susceptible mouse strain Balb/c (but not in the resilient strain C57BI/6), early life stress elicits biphasic changes in HDAC expression and histone modifications during postnatal development that trigger several post-translational modifications of histone proteins.

From those, increased expression of histone H4 acetylated at lysine residue K12 is the most prominent epigenetic phenotype in adulthood. Low doses of the HDAC activator theophylline effectively reduced the expression of acetylated H4K12 protein when administered during adolescence, and worsened two prominent behavioral phenotypes that characterize IMS Balb/c mice. In contrast, the antidepressant drug fluoxetine did not only ameliorate the severity of these behavioral phenotypes resulting from IMS exposure (Bhansali et al., 2007), but also augmented the histone modifications elicited by early life stress. These findings indicate that the reduced HDAC activity in IMS Balb/c mice is a positive adaptive process that ameliorates the severity of abnormal emotional behaviors.

In the forebrain neocortex of IMS Balb/c mice, changes in HDAC expression and histone modifications emerge in early adolescence (P21), but then exhibit a biphasic developmental pattern. Expression of HDACs 1, 3, 8, and 10 was increased during early (P21-P28) adolescence, but persistently decreased afterwards. Although HDAC7 was consistently decreased between P21 and P60, the increased expression of the HDACs 1, 3, 8, and 10 predominated in modifying the histone H4 acetylation phenotype measured at P21, i.e., acetylation of histone H4K12 and H4K8 was decreased and H4K5 and H4K16 acetylation was unaltered. Conversely, at P60, the expression of all 5 HDACs was lower compared to controls, and acetylation of histone H4K5, H4K8, and H3K12 was significantly increased. Nevertheless, at both developmental ages (P21 and P60), the effect of IMS was largest for the expression of acetylated histone H4K12, which is thought to play a unique role in orchestrating gene expression (Kwang et al., 2007) and has already been shown to be crucially involved in regulating the expression of hippocampal genes that are required for the formation of memories (Peleg et al., 2010).

Although the developmental profile of modified histone expression was not measured between P21 and P59, the data shown in FIGS. 1 and 2 clearly show that HDAC expression and expression levels of acetylated histone H4 parallel each other. The mechanism by which early life stress affects only distinct HDACs is presently unknown. Equally unknown is the reason for the biphasic nature of the HDAC-triggered histone modifications in IMS Balb/c mice. Of note, a similar biphasic change in gene expression has also been found for the glucocorticoid receptor that, in the forebrain neocortex of IMS Balb/c mice, is expressed at higher levels during adolescence but at reduced levels in adulthood (Navailles et al., 2010). Although one possibility could be that early-life stress-induced changes in gene expression during early to mid-adolescence affect critical developmental processes of the forebrain neocortex that ultimately lead to the establishment of the adult phenotype precipitated by early life stress, it was found that treating IMS Balb/c mice with the HDAC inhibitor SAHA between P21 and P35 (which prevented the decreased acetylation of histone H4 proteins) did not prevent the development of the adult epigenetic phenotype, thus, making a dependence of the developing adult phenotype from the P21 phenotype unlikely.

IMS Balb/c mice also exhibited increased expression of dimethylated histone H3K9. In contrast to the biphasic changes in histone H4 acetylation, however, increased dimethylation of H3K9 protein was only observed in adulthood. Although dimethylated histone H3 is considered a marker of transcriptional repression, there is also evidence that histone H3 methylation facilitates histone acetylation (Wang et al., 2009; Zhang et al., 2004). Whether there is indeed a functional link between histone H4 acetylation and histone H3 dimethylation, especially between histone H4K12 acetylation and histone H3K9 dimethylation in the adult brain, remains to be demonstrated.

Although altered expression of three class I and two class II HDACs in IMS Balb/c mice was found, the weight of the evidence suggest that the IMS-triggered changes in histone modifications can largely be accounted for by the class I HDACs 1, 3, and 8. The most predominant IMS-specific change in histone H4K12 acetylation was reversible by a low dose of theophylline that has been shown to activate HDAC1 and HDAC3, but not HDAC2 (Ito et al., 2002) without exerting other, possibly confounding, effects on adenosine receptors or phosphodiesterase inhibitors. In addition, HDAC8 has been shown to be crucial in deacetylating histone H4 (and also H3; see Lee et al., 2004). However, whether class I-selective HDAC inhibitors can recapitulate the entire chromatin modification phenotype resulting from IMS remains to be tested. Of note, despite their high degree of sequence similarity (de Ruijter et al., 2003), HDAC1, but not HDAC2, was affected by IMS. This is not surprising since previous studies on the role of HDAC2 in hippocampal memory and synaptic plasticity have already shown that HDAC1 and HDAC2 are not functionally redundant (Guan et al., 2009).

There are two major roles of HDACs. One is to remove the acetyl groups added by histone acetyltransferases at active genes during transcriptional initiation and elongation, the other is to maintain a reduced level of histone acetylation at, and to prevent RNA polymerase II from binding to, silent genes (Wang et al., 2009). In adult IMS Balb/c mice, histone H4K12 was hyperacetylated, and increased acetylation also occurred for histones H4K5 and H4K8. Such epigenetic markers suggest increased transcriptional activity, and the here-identified histone modifications open the door for chromatin immunoprecipitation-guided identification of the affected genes.

Do the changes in histone modifications described here contribute to the IMS specific behavioral phenotype or do they ameliorate the severity thereof? The latter possibility is supported by results shown in FIG. 5 indicating that two prominent behavioral phenotypes found in IMS Balb/c mice, namely increased anxiety and increased passivity in stressful environments (Mehta and Schmauss, 2011), are potentiated by a concentration of theophylline that, although effectively reversing the effect of IMS on histone H4K12 acetylation, has no demonstrated effects on adenosine receptors or phosphodiesterase inhibition (FIG. 4) and, as further shown in FIG. 6, had no behavioral effects in SFR controls. In contrast, adolescent fluoxetine has not only been shown to be effective in improving the emotional phenotype of IMS Balb/c mice (Bhansali et al., 2007), it also increased the expression of total H3 and H4 histones, an effect that resulted in a further increase of acetylated H4 (and also histone H3) protein expression in IMS Balb/c mice (Table 5). This finding led to the test of whether, in non-stressed (SFR) mice, the antidepressant efficacy of fluoxetine can also be increased when HDAC activity is pharmacologically reduced. Indeed, when SFR mice were only treated with fluoxetine during adolescence, their behavioral responses to EPM and FST exposure were unaltered. In contrast, SFR mice treated with the HDAC inhibitor SAHA and fluoxetine during adolescence exhibited significantly less anxiety-like and depression-like behaviors in the EPM and FST tests, respectively (FIG. 6). Moreover, the combined treatment of SAHA and fluoxetine also recapitulated most of the effects of fluoxetine on the expression of histone proteins detected in IMS Balb/c mice, namely increased expression of total histone H4 protein along with increased expression of acetylated histone H4K12, and increased expression of trimethylated histone H3 protein, indicating that these changes in histone expression are critically involved in potentiating the behavioral responsiveness to fluoxetine treatment.

Altogether, the data suggest that reduced HDAC activity and the resultant histone modification are a positive adaptation developing in IMS Balb/c mice, at least in terms of the severity of the emotional phenotype that results from early life stress exposure. Moreover, this epigenetic adaptation enhances the antidepressant effects of adolescent fluoxetine treatment.

The present study is the first to demonstrate a global increase in histone acetylation in the forebrain neocortex of a stress-susceptible strain of mice exposed to early life stress that is paralleled by reduced HDAC expression found in this anatomic region but not in the hippocampus or striatum. It is, at present, not clear whether these epigenetic changes are specific for the forebrain neocortex or whether other anatomic regions implicated in stress responses, such as the amygdala or the hypothalamus, also exhibit decreased HDAC expression after early life stress exposure. Nevertheless, it is evident that HDACs affected by early life stress as well as the anatomic region prominently affected by reduced HDAC expression differ from those previously linked to adult stress (for review see Fischer et al., 2010).

There is one important common theme to the effects of early life and adult stress on HDAC activity, namely that reducing HDAC activity is an adaptive phenomenon with antidepressant effects. This is supported by the present findings as well as by earlier studies that employed chronic adult stressors and examined the effect of adult antidepressant treatment (Tsankova et al., 2006; Wilkinson et al., 2009). However, it must be stressed that reduced HDAC activity has positive adaptive effects on the behavioral phenotype only in chronically stressed animals. In fact, the data indicate that reducing HDAC activity has deleterious effects on the behavior of SFR control mice, i.e., SFR mice treated with SAHA during adolescence (without fluoxetine co-treatment) exhibited increased anxiety-like behavior in the EPM test and increased depression-like behavior in the FST (FIG. 6). Thus, equal behavioral responses of SFR and IMS mice to EPM and FST exposure depend upon different epigenetic landscapes, with IMS mice requiring a greater ratio of expression of acetylated over non-acetylated H4 histones. Interestingly, the same holds true for the behavioral responses to fluoxetine treatment that are enhanced when increased expression of acetylated histones occurs. Yet, disruption of either the epigenetic phenotype of stressed animals (FIG. 5) or the normal epigenetic signature of non-stressed animals (FIG. 6) leads to the same abnormal anxiety- and depression-like behavioral phenotype.

### Example 10

### Enhanced Antidepressant Effect of Fluoxetine is Due to Increased Histone Expression

As set forth above, H4K12 hyperacetylation can be reversed by treating IMS Balb/c mice with the HDAC activator theophylline during adolescence (P35 to P59; see Table 7 below). However, this treatment worsened the emotive behavioral phenotype of adult IMS Balb/c mice, indicating that this histone modification decreases the severity of the adult emotive phenotype (see FIG. 5). In contrast, chronic adolescent treatment with the antidepressant drug fluoxetine improved not only the emotive behavior of IMS Balb/c mice (Bhansali et al., 2007), but also potentiated the histone hyperacetylation resulting from early life stress, an effect accompanied by a significant increase in the expression of total histone H3 and H4 proteins (Table 7). As shown further below, the same is observed in non-stressed Balb/c mice that are co-treated with the HDAC inhibitor SAHA and fluoxetine (see FIG. 8).

TABLE 7
Effect of Adolescent Theophylline (tph) and Fluoxetine (fluox) on Histone
Modifications in IMS Balb/c Mice^{b}

| | HDAC | | | | | | | | | |
|---|---|---|---|---|---|---|---|---|---|---|
| | mRNA | H4 | acH4K12 | acK4K8 | acH4K5 | acH4K16 | H3 | acH3K9 | H3K9me2 | H3K4me3 |
| Tph | = | = | Decreased p < 0.01 | = | = | = | = | = | = | = |
| fluox | = | Increased | Increased | Increased | Increased | = | Increased | Increased | = | Increased |
| | | p < 0.001 | p < 0.001 | p < 0.02 | p < 0.05 | | p < 0.001 | p < 0.001 | | p < 0.03 |

^{b}Data (n = 5/group) were compared to non-treated IMS Balb/c mice (two-tailed t tests);
= indicates unaltered expression

These findings suggest that the histone modification phenotype influences the antidepressant efficacy of fluoxetine, and that a 5-HT-dependent mechanism is responsible for the drug-induced stimulation of histone expression. Note that, in the visual cortex, there is precedent evidence that the effects of fluoxetine on the reinstatement of adult cortical plasticity are also due to chromatin modifications that are triggered by serotonin acting on 5-HT1A receptors (Vetencourt et al., 2008). Hence, whether the stimulatory effects on histone expression enhance the efficacy of antidepressant drugs and, whether these effects require increased serotonergic signaling, are of significance for the future of developing more efficacious antidepressant drugs.

In IMS mice, fluoxetine increased the expression of total H4 and H3 proteins and, thereby, further increased expression levels of acetylated histones that were already elevated in IMS mice at baseline (Table 3). In SFR mice, however, fluoxetine exerted no effect on histone expression (FIG. 7). These findings indicate that fluoxetine stimulates histone H4 expression only in mice with reduced HDAC activity. This effect is dependent upon enhanced serotonergic signaling and it is a critical determinant of antidepressant efficacy.

It remains to be investigated whether the stimulatory effect on histone expression is dependent upon enhanced serotonin (5-HT) signaling. SSRIs like fluoxetine are thought to exert their antidepressant effects through adaptive changes in 5-HT systems that are triggered by increased extracellular 5-HT concentrations. For comparison, one antidepressant drug that is structurally more similar to tricyclic antidepressants, namely tianeptine, has pharmacological properties that are opposite from those of SSRIs: tianeptine increases the Vₘₐₓ of 5-HT uptake without marked changes in extracellular 5-HT levels (McEwen et al., 2010, Mennini et al., 1987, Park et al., 1992). Moreover, chronic tianeptine treatment does not lead to desensitization of 5-HT1A autoreceptors or alters the activity of postsynaptic 5-HT receptors, but it decreases stress-induced glutamate release. Also these effects are opposite from those exerted by SSRIs (McEwen et al., 2010, Park et al., 1992).

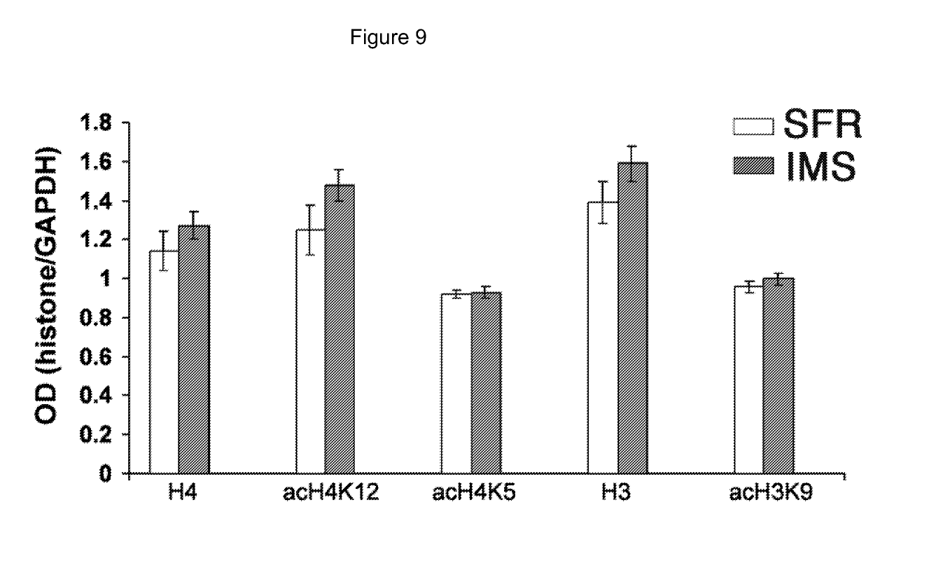

Strikingly, as shown in FIG. 9, in contrast to the effect of adolescent fluoxetine in SAHA co-treated SFR mice, adolescent co-treatment with SAHA and tianeptine increased histone H4 and aCH4K12 expression only minimally, and none of these effects reached significance (the effect on aCH4K12 expression is likely due to SAHA alone). This treatment also did not significantly affect histone H3 and aCH3K9 expression (FIG. 9) and exerted no effect on EPM and FST behavior (not shown). These data suggest that the effect of the SSRI fluoxetine on histone H4 expression is critical for antidepressant efficacy.

These are the first findings of persistent changes in histone modifications that result from early life stress and that influence emotive behavior and antidepressant treatment response. They were detected in the stress-susceptible strain Balb/c, but not in the resilient strain C67BI/6. Moreover, in young adults, they are restricted to the forebrain neocortex of IMS Balb/c mice, a finding similar to the persistent changes in expression of several genes that the inventors have previously reported to result from early life stress exposure (Schmauss et al., 2010, Navailles et al, 2010, Bhansali et al., 2007).

Thus, these findings indicate that fluoxetine stimulates histone H4 expression only in mice with reduced HDAC activity and that HDAC activity is a critical determinant of the antidepressant efficacy of drugs that enhance serotonergic signaling.

The idea that patients who respond to fluoxetine and those who do not respond differ in their epigenetic phenotypes is completely novel and directly testable. Moreover, targeting the epigenetic phenotype that confers resistance to SSRI treatment promises significant enhancement of antidepressant efficacy of SSRIs. This is further supported by data shown below.

### Example 11

### The Effectiveness of Class I HDACi Inhibition in Enhancing the Antidepressant Efficacy of Fluoxetine

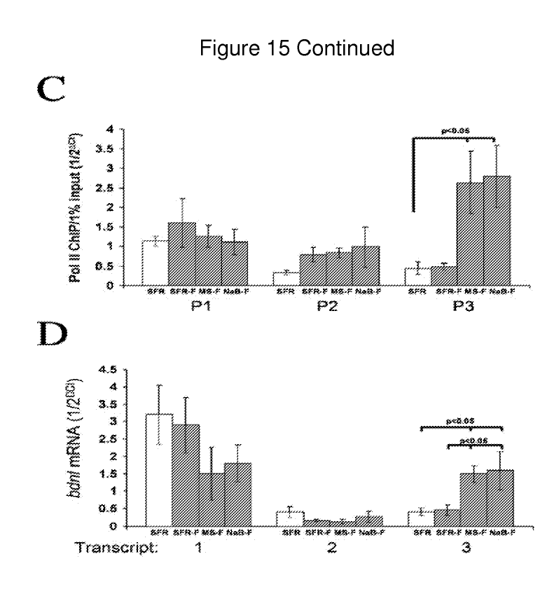

The following experiments were performed to show that class I HDAC inhibition is sufficient to enhance the antidepressant efficacy of the SSRI fluoxetine, especially inhibitors of HDACs 1 and 3. Briefly, studies were performed on non-stressed Balb/c mice that were treated during adolescent development (postnatal age P35 to P59) with MS-275 (a class I HDACi that inhibits HDAC 1 and HDAC 3 with an IC₅₀ of 0.51 and 1.7 μM, respectively) and fluoxetine (16 mg/kg/day). As shown in FIG. 15A, compared to vehicle treated and fluoxetine-only treated Balb/c mice, mice co-treated with MS-275 (15 μM/day; labeled MS-F in FIG. 15) and fluoxetine exhibited significantly reduced immobility in the forced swim test (FST), indicating that this co-treatment elicited antidepressant-like effects that were not achieved with fluoxetine alone. Additional experiments were performed on fluoxetine-treated mice that were co-treated with sodium butyrate (NaB; 6 g/kg/day; labeled NaB-F in FIG. 15) which, in addition to inhibiting HDACs 1 and 3 (IC₅₀: 0.3 and 0.7 mM, respectively), also inhibits class II HDAC 7 (IC₅₀: 0.3 mM). As shown in FIG. 15A, the performance of these mice in the FST was indistinguishable from the performance MS-F mice. Hence, HDAC 1 and 3 inhibition is sufficient to enhance the antidepressant effects of fluoxetine in the FST.

Additional chromatin immunoprecipitation (ChIP) studies focused on the effect of these co-treatments on the enrichments of the histone marker aCH4K12 (a marker of active gene transcription) at different promotors of the gene encoding brain-derived neurotrophic factor (BDNF). This gene was targeted because it has been shown that efficacious fluoxetine treatment increases expression of the BDNF gene. As shown in FIG. 15B, BDNF promotors P1 and P3 experience higher enrichment of aCH4K12 in fluoxetine-only and fluoxetine/MS-F and fluoxetine/NaB-F mice. However, as shown in FIG. 15C, enhanced density of the actively-transcribing form of RNA Polymerase II (Pol II) is only detected for the P3 promotor of the BDNF gene. Moreover, the density of Pol II associated with the P3 promotor was only increased in co-treated mice, and not in fluoxetine-only treated mice.

The results shown in FIG. 15C are consistent with results in FIG. 15D showing that neither BDNF transcript variants 1 and 2 differ significantly between the four treatment groups, and that only the BDNF transcript variant 3 mRNA is increased in co-treated mice, but not in fluoxetine-only treated mice. Again, increased Pol II enrichment at the P3 promotor and increased expression of BDNF transcript variant 3 expression is highly similar in fluoxetine/MS-F and fluoxetine/NaB-F mice, indicating that HDAC1 and HDAC3 inhibition is sufficient to exert the epigenetic response that has a functional impact on BDNF expression. Altogether, these data provide strong support for the enhancing effects of HDAC 1 and 3 inhibition on the antidepressant efficacy of the SSRI fluoxetine.

### Example 12

### IMS-Triggered Histone H4 Hyperacetylation Affect Cognitive Functioning

The inventor has found that IMS Balb/c mice exhibit Working Memory (WM) and attention-set-shifting deficits in young adulthood (Mehta and Schmauss, 2011). These cognitive functions are governed by the medial prefrontal cortex (mPFC), i.e., an anatomic region known for discrete structural abnormalities that are triggered by early life stress exposure (Braun et al., 2000, Helmeke et al., 2008, Pascual et al., 2007). Yet, whether the epigenetic response to early life stress affects these cognitive functions in IMS Balb/c is still unknown.

To date, almost all studies on the modulation of cognitive functions by epigenetic regulators (i.e., DNA methylation and histone modifications) have focused on hippocampal, memory-related function (Barrett et al., 2008, Day et al., 2011, Guan et al., 2009, Peleg et al., 2010). In contrast, very little is known about the role of an epigenetic control of gene expression during cognitive task performances that depend upon the PFC. Only recently, a human study demonstrated the first link between an epigenetic marker of the Val/Val-COMT allele (that creates a CpG methylation site) and prefrontal cortical activity during WM task performance. This study showed that stress-related hypomethylation at this CpG methylation site associates with decreased response accuracy in the WM task and less efficient PFC activation (Ursini et al., 2011).

The hypothesis that specific changes in gene expression in the mPFC that result from the increased acetylation of histone H4 proteins, especially acetylation of histone H4K12 (aCH4K12), directly affect WM and attention set-shifting, will be tested. Different complementary approaches will be taken. First, whether a reversal of the most prominent histone modification found in IMS Balb/c mice (hyperacetylation of histone H4K12) affects WM and attention set-shifting test (ASST) performance will be tested. Second, whether IMS Balb/c mice express higher levels of acetylated H4 proteins predominantly in the mPFC will be examined.

As shown in FIG. 4, the most prominent IMS-triggered increase in aCH4K12 expression could be significantly decreased by chronic treatment of adolescent IMS Balb/c mice with theophylline. Theophylline also had significant effects on the emotive behavior of these mice. It elicited increased anxiety in the Elevated Plus Maze (EPM; FIG. 5A) and increased immobility in the Forced Swim Test (FST; FIG. 5B). At the concentration administered (32 mg/kg/day) theophylline mainly activates the class I HDACs 1 and 3 (Ito et al., 2002).

Whether decreased expression of aCH4K12 in theophylline-treated IMS Balb/c mice also affects their performance in the WM and ASST tests will be tested. The mice will be exposed to the IMS protocol (a daily 3-hour separation of pups from their mothers from postnatal age P2 to P15). Standard-facility-reared (SFR) mice serve as controls. All mice will be weaned at P28. Half of the mice (both SFR and IMS, randomly selected from different litters) will receive theophylline (32 mg/kg/day) via the drinking water, starting at postnatal age P35 and ending at P59. The other half receives regular drinking water. Starting at P60, all mice receive regular drinking water and will be food-restricted gradually until they loose about 10% of the starting body weight. They will then be tested either in the ASST or trained for the WM test. Thus, since mice are no longer receiving theophylline at the time of testing, the possibility that other pharmacological properties of the drug (i.e., adenosine receptor antagonism, phosphodiesterase inhibition) affect cognitive functions is eliminated.

The ASST is the rodent equivalent of the Wisconsin Card Sorting test used in humans, but relies on rodent-specific stimulus dimensions (odor, texture) that guide correct response selections under changing rules. Briefly, mice proceed through the ASST as follows. After simple discrimination learning (odor vs. texture), mice go through a series of compound discriminations whose rules are guided by one of the two stimulus dimensions (odor or texture) and, after successful completion of an intradimensional set-shifting test phase (IDS), they are required to engage in extradimensional shifts (EDS) of attention, i.e., if odor guided in the IDS phase, it will now be texture that guides correct response selection in the EDS. Finally, the rules of the EDS will be reversed. Six consecutive correct trials end each phase of the ASST.

The WM test is a delayed alternation test of spatial working memory and is performed in a T-maze. Briefly, mice are trained for alternative arm entries over a period of 7 to 10 days. After they reach at least 70% correct (left-right alternating) arm entries on two consecutive days, they are tested for their ability to hold the spatial WM “online” during inter-trial delay periods of 15, 20, and 30 seconds.

For both tests, data are compared by repeated measures ANOVA. The statistical details and necessary group sizes (usually 8 mice per group and treatment condition) are described in Mehta and Schmauss (2011). Note that there were no sex differences in the two cognitive tasks (Id.). Thus, an equal number of males and females will be used. Moreover, brains of mice with and without theophylline treatment (both SFR and IMS) will be collected for protein extraction to control for the expression levels of aCH4K12 protein in the frontal cortex by Western blotting.

If the epigenetic marks of early life stress indeed affect WM and attentional set-shifting, but not recognition memory and reversal learning, increased expression of acetylated histone H4 protein should be prominent in anatomic regions that govern these function, i.e., the pre- (Prl)- and infralimbic (IL) subregions of the mPFC (Aggleton et al., 1995, Birrell et al., 2000). To test this, quantitative anatomic studies will be performed on brain sections of SFR and IMS Balb/c mice that are immunolabled with one of four antibodies: anti-aCH4K12, anti-aCH4K5, anti-aCH4K8, and anti-H3me2 (Millipore). The expression of these histone proteins is increased in IMS Balb/c mice at P60 (Table 3).

The experiments will be conducted in two stages. The first stage involves a quantitative assessment of immunoreactivity across anatomically defined fronto-parietal regions of SFR and IMS Balb/c mice. This will identify the anatomic boundaries of increased aCH4 protein expression in IMS mice and hence, determine the region of interest (ROI) for stage 2. To expedite the identification of the ROIs, densitomertric measures will be obtained (Navailles et al., 2010). Briefly, immunolabeled sections (40 μm, collected at an intraseries interval of 200 μm) are photo-documented (Improvision Openlab) and then processed using Adobe Photoshop. The expression of aCH4 immunoreactivity (K5, K8, and K12) will be analyzed using NIH ImageJ software by measuring the pixel densities in each ROI (orbital frontal cortex, mPFC, motor, somatosensory and parietal cortex) in three consecutive sections. The digitized images are converted to 8-bit grey scale, and calibrated using the pixel inverter function. Each region of interest will be delineated using bregma coordinates, the mean pixel density of the selected area will be measured, and the mean pixel density value of the background area surrounding the ROI will be systematically subtracted. The average pixel density obtained for each ROI will be calculated and expressed as the mean pixel density per region and animal group.

In stage 2, the number of immunolabled cells within subregions of the ROI will be estimated via systematic-random sampling using the optical fractionator (West et al., 1991), because the behavioral data (Mehta and Schmauss, 2011) as well as previous structural studies on brains of IMS rodents (Braun et al., 2000, Helmeke et al., 2008, Pascual et al., 2007) suggest strongly that the mPFC is the prime candidate ROI among all fronto-parietal regions. Moreover, results from the Western blots suggest further that increased aCH4K12 expression will be the most prominent mPFC phenotype in IMS mice. Thus, if confirmed with the stage 1 experiments, stereological measures will be taken from three (IL) or six (Prl and AC subregions of the mPFC) sections from each series. Optical disector frame and counting grid sizes of 30 to 45 and 75-100 μm², respectively, should permit systematic-random sampling of >3 neurons within an 8 μm focusing range for each sampling field. Sampling parameters will be set such that at least 200 neurons per region are sampled in the cases with lowest aCH4 expression, and measures are taken for superficial (II/III) and deep layers (V/VI) of mPFC regions. Intra-sample coefficients of error (CE), calculated as described in Schmitz (2000), should be less than 0.05 and no significant differences in CE values should exist between groups. All regions are sampled at high magnification (100×) in Koehler illumination conditions. The volume of the different laminar domains of three mPFC regions will be estimated using the Cavalieri principle. The amount of aCH4K12-labeling will be expressed as the number of aCH4K12-immunoreactive neurons per 0.1 mm³ so that the net expression per region can be compared between groups. Densities of aCH4K12-labeled neurons will be obtained by dividing the mean number of aCH4K12-labeled neurons of each group by the average volume of the corresponding region. Similar measures would be obtained for aCH4K5- and aCH4K8- or H3me2-immunolabled cells if stage 1 experiments point to differences in their expression between groups in the mPFC and, if other ROIs are also defined in stage 1, stage 2 experiments will be performed, and the sampling parameters will be adjusted accordingly.

As summarized above, although epigenetic mechanisms controlling learning and memory-related cognitive functions have long been proposed, little is known about how epigenetic mechanisms affect executive cognitive functions governed by PFC circuitries. The study on IMS Balb/c mice is especially powerful for elucidating such role in prefrontal cortical functions because young adult IMS Balb/c mice have WM and set-shifting deficits, but no deficits in learning and memory-related cognitive function (Mehta and Schmauss, 2011). Because the altered epigenetic landscape of IMS Balb/c mice is anatomically restricted to frontal cortical regions and, because this epigenetic landscape of IMS Balb/c mice were analyzed in great detail, candidate histone proteins with distinct post-translational modifications will be examined for their roles in modulating WM and attentional set-shifting phenotypes.

There are two possible outcomes of the behavioral experiments disclosed above. Similar to the effect of adolescent theophylline on the emotive behavior of IMS Balb/c mice, theophylline may worsen the WM and set-shifting deficits. Alternatively, the H4K12 hyperacetylation is, in fact, the cause of the cognitive deficits, theophylline treatment will improve these cognitive functions. In either case, that nature of the cognitive deficits of IMS Balb/c mice suggests that especially the IL/PrL subregions of the mPFC exhibit significantly elevated histone H4 acetylation, and only the stereological measures disclosed above can demonstrate this directly.

This is of importance because, in humans, deficits in WM and attention are indicators of risk for (Erlenmeyer-Kimling et al., 2000, Gottesman et al., 2001) and endophenotypes of manifest schizophrenia and mood disorders (Park et al., 1992, Nuechchterlein et al., 2004, Marazziti et al., 2010, Zihl et al., 1998), i.e., two disorders for which early life stress is a common environmental risk factor. Although both diseases have different genetic susceptibility and different disturbances in monoaminergic, cholinergic, GABAergic, or glutamatergic innervation of the PFC, the occurrence of the same cognitive deficits in IMS Balb/c mice indicate that there exists at least one common final path that can lead to these deficits, a path triggered by environmental factors that alter epigenetic marks in the PFC. Thus, the significance of the studies resides in the rigorous investigation of the role of an epigenetic modulation of mPFC function. Novel treatments of the cognitive deficits of stress-related mental disorders that target the epigenetic phenotype will be available.

### Example 13

### The Effect of Early Life Stress on Aging

Young adult IMS Balb/c mice exhibit deficits in specific cognitive functions (Mehta and Schmauss, 2011). Three cognitive tests designed for rodents were used. One test measured Social Recognition Memory (SRM), another tested spatial Working Memory (WM), and the third was the Attention-Set-Shifting Test (ASST) that taxes three additional cognitive domains, namely associative learning, attention set-shifting, and reversal learning. Consistent with the changes in gene expression and histone modifications that are restricted to the forebrain neocortex of IMS Balb/c mice, it was found that deficits in functions governed by the medial prefrontal cortex (mPFC), namely spatial WM deficits and deficits in extradimensional set shifting. This is of particular interest because these two cognitive deficits are strong endophenotypic indicators of prefrontal cortical dysfunctions found in several major mental diseases including depression (Aggleton et al., 1995, Birrell et al., 2000). In contrast, associative learning, short-term memory (a hippocampal/perirhinal function) (Davis et al., 2010, Brown et al., 2001), and reversal learning (a function of the orbital frontal cortex) (McAlonan et al., 2003) were unaffected by ELS, at least in young adults.

In IMS Balb/c mice, cognitive deficits associated with reduced mPFC function (spatial WM, set-shifting), are the first cognitive deficits to emerge (Mehta and Schmauss, 2011). Consistent with previous findings (Brunson et al., 2005), despite the presence of these deficits, P60 IMS mice exhibit no cognitive deficits indicative of hippocampal/perirhinal or orbital frontal cortical dysfunction (Mehta and Schmauss, 2011). This is not surprising because at P60, IMS mice exhibit reduced class I HDAC activity (HDACs 1, 3 and 8). In fact, HDAC3 is a negative regulator of long-term memory (McQuown et al., 2011), and class I HDAC inhibitors reverse contextual memory deficits in a mouse model of Alzheimer's disease (Kilgore et al, 2010). Moreover, P60 IMS Balb/c mice express elevated levels of aCH4K12, but reduced aCH4K12 expression is associated with age-related memory impairments (Peleg et al, 2010).

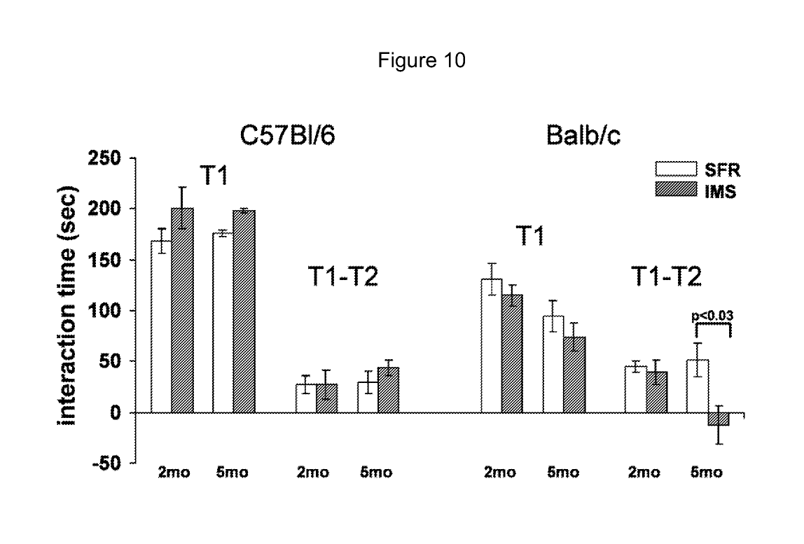

There is, however, also a late-onset progression of cognitive dysfunctions after early life stress exposure. In rats exposed to early life stress, Brunson et al. found that synaptic and behavioral measures of hippocampal function deteriorate at mid age (Brunson et al., 2005). Moreover, as shown in FIG. 10, IMS Balb/c exhibited deficits in social recognition memory, but not IMS C57BI/6 mice, that emerge at 5 months of age.

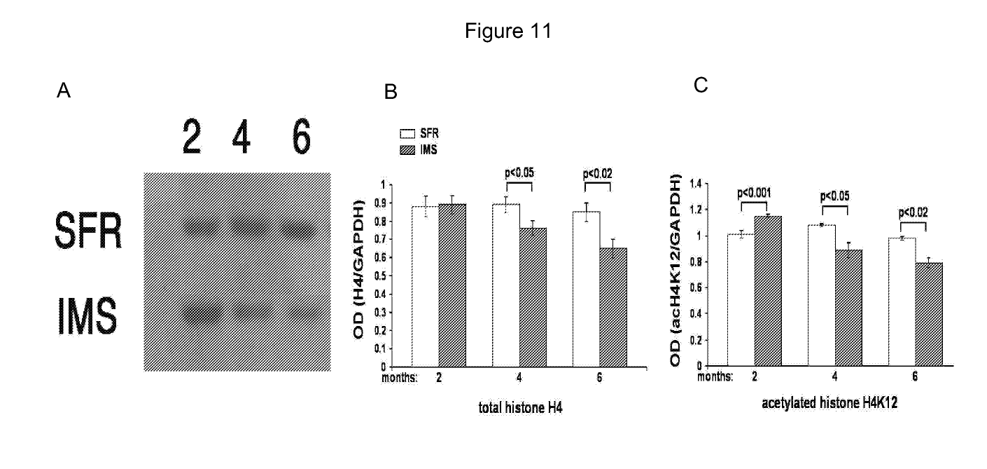

This finding is of significance because aging-related memory deficits manifest predominantly as declarative/episodic memory deficits (such as recognition memory) and working memory deficits. The two brain regions that govern these functions (hippocampus/perirhinal cortex and PFC), are particularly susceptible to aging (Boss et al., 2011, Hara et al, 2011), and it is thought that dysregulation of epigenetic control drive these age-related cognitive dysfunctions (Penner et al., 2010). Indeed, as shown in FIG. 11, a progressive decline in frontal cortical histone H4 and aCH4K12 expression was observed in 4 and 6 months old IMS Balb/c mice.

It is known that decreased total levels of H3, H4, and 2A lead to irregular spacing of nucleosomes, a progressive loss of chromatin density and thus, loosening chromatin structure (Feser, 2011). Moreover, in yeast, a causal link between decreased H4 expression and aging has already been shown (Feser et al., 2011). Also, an approximately 50% decrease of H3 and H4 expression was found in aging human fibroblasts (O'Sullivan et al., 2010), and a bulk reduction in histone H4 acetylation was found in the aging rat cerebral cortex (Ryan et al., 1972). Hence, because histone depletion and the resultant chromatin-dependent transcriptional misregulation are general phenomena of aging, it is expected that early life stress accelerates the onset of these molecular signs of aging, and that this accounts for the emergence of new cognitive dysfunctions in mid-aged IMS Balb/c mice.

### Example 14

### Histone Expression, Cognitive Function Deterioration, and Aging

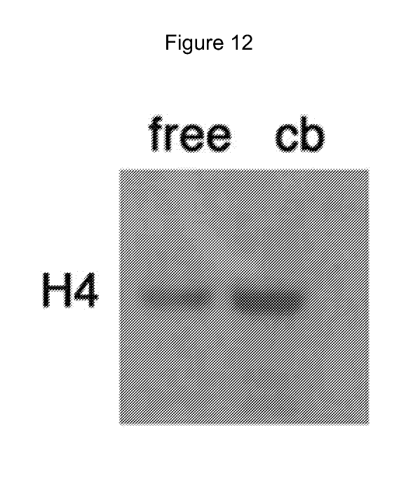

To follow up on findings shown in FIG. 11, histone expression in SFR and IMS Balb/c mice at 2, 3, 5, 8, and 12 months of age will be analyzed. Because it is possible that the constant hyperacetylation of histone H4 in adolescent and young adults eventually triggers the histone loss during aging, IMS Balb/c mice treated with theophylline during adolescence to lower their aCH4K12 expression will be included (FIG. 5). Moreover, because chromatin-bound histone proteins are constantly replaced with free histones, and because lower concentration of free histones limits the extent of histone exchanges, chromatin fractionations will be performed to compare free and chromatin-bound histone expression (see FIG. 12). In Western blots of free and chromatin-bound proteins, antibodies directed against total histone 2A, H3, and H4 as well as antibodies directed against the acetylated and methylated histone variants listed in Table 3 will be used. Because it is also desirable to test whether histone loss remains restricted to the anatomic region with increased aCH4 protein expression at 2 months of age or, whether it also spreads to other anatomic regions such as the hippocampus (as suggested by the behavioral data shown in FIG. 10), these studies on dissected mPFC and hippocampal/perirhinal tissues will be conducted.

One characteristic phenotype of IMS Balb/c mice is their increased response to adult stress. Thus, even mild adult stress could further accelerate the emergence of molecular and behavioral signs of aging in these mice. To test this, IMS mice that were exposed to adult stressors will also be examined. An effective, yet chronic ultra-mild stress (CUMS) paradigm (Francis et al., 2003) that lasts for a period of 1 week will be used. Briefly, starting at 2 months of age, SFR and IMS Balb/c mice will be exposed to various mild stressors, such as repeated periods of a 30 degree cage tilt, confinement to small cages, 2 hour period of paired housing with an unfamiliar mouse, one overnight period of difficult access to food, one period of continuous overnight illumination, and one overnight period in a soiled cage, followed by a reversal of the light dark cycle for 2 days. This one-week CUMS exposure can be repeated at 3 and 4 months of age.

The ends of linear chromosomes, so-called telomeres (tandem repeats of TTAGGG sequences), play an essential role in the stable maintenance of eukaryotic chromosomes within a cell by binding to structural proteins that cap the end of chromosomes and prevent nucleolytic degradation. Decreased histone H4 expression has been shown to alter transcription of genes near telomeric regions, and telomeres progressively shorten with increasing age (Wyrick et al., 1999). In the CNS, microglia are the only cell type with significant mitotic potential and thus, they are susceptible to telomere shortening. Indeed, Flanary and Streit (2003) have shown that telomere shortening occurs in rat cerebellum and cortex with increasing age (detectable already at 5 months) and that low telomerase activity in these tissues maintains preferentially the shortest telomeres while the longest telomeres shorten more rapidly. This finding has indeed been attributed to microglial cell division (Flanary and Streit, 2005).

Telomere lengths in mPFC and hippocampal/perirhinal DNA extracted from the groups of mice described above will be measured. The Telo TAGGG Telomere Length Assay kit (Roche), which relies on Southern blots of terminal DNA restriction fragments of frequent cutters that do not cut telomeres and subtelomeric sequences (Hinf I, Rsa I), will be used. Blotted DNA is then hybridized to a digoxigenin (DIG)-labeled probe that specifically recognizes telomere repeat sequences. The bound probe is visualized by a chemiluminescent substrate for alkaline phosphatase (CDP-Star), and the average telomere length is determined using molecular weight standards.

The data shown in FIG. 10 support the hypothesis that aging-related cognitive deficits, such as deficits in recognition memory, occur earlier in IMS Balb/c mice compared to their SFR controls. The experiments described above will address the question whether only the mPFC (top down control), or also the hippocampus/perirhinal cortex (object recognition and encoding of familiarity), is affected by this accelerated aging process. The findings shown in FIG. 10 will be further investigated. A behavioral test battery will be used to monitor the behavior of IMS and SFR mice at 2, 3, 5, 8, and 12 months of age. This test battery consists of two tests of emotive behavior (the EPM and FST) and three cognitive tests, namely the SRM test, and the spatial WM and ASST tests. While IMS Balb/c mice at 2 months of age only exhibit WM and attention set-shifting deficits and no deficits in SRM, 5 months-old IMS mice exhibit significant deficits in SRM (FIG. 10). Given that both WM and episodic memory are cognitive functions that are most sensitive to aging, it is expected that the WM deficits of IMS Balb/c mice also progressively worsen between 5 and 12 months of age and that this is paralleled by histone depletion during this time. Moreover, if the histone depletion spreads across the forebrain, other deficits, such as deficits in discrimination and reversal learning, will also be detected.

In young adult IMS Balb/c mice (P60), the expression levels of total histone H3 and H4 protein is unaltered (Table 3). Rather, a significantly larger proportion of histone H4 protein is acetylated. This hyperacetylation phenotype, however, is not maintained throughout adulthood. The data uncovered a progressive histone H4 depletion in IMS Balb/c mice at 4 and 6 months of age that also leads to decreased expression of aCH4K12 protein. Moreover, a new cognitive deficit emerges during this time, namely impaired recognition memory. Since both histone depletion and late-onset episodic memory deficits are strong molecular and behavioral indicators of aging, these findings point for the first time to a link between early life stress exposure and accelerated aging. It is anticipated that these studies will provide widely accepted molecular (histone levels, telomere lengths) and behavioral (SRM and WM deficits) evidence for this.

It is expected that the experiments disclosed herein will illustrate that early life stress exposure affects later development in two phases: an early-onset phase in young adults with partially adaptive changes in the post-translational modification of histone H4 protein and cognitive deficits delimited to two distinct executive functions of the mPFC, and a late-onset phase of accelerated emergence of molecular and cognitive signs of aging. It is further expected that the late-onset phase will be characterized by histone depletion and spreading of deficits to other (memory-related) cognitive domains, a process that is possibly triggered by the constant hyperacetylation of histone H4 in younger animals.

There is an intriguing possibility that reversing age-related histone depletion may delay the onset of aging-related cognitive deficits, and the experiments disclosed herein speak directly to this.

Three important additional questions are also addressed by the experiments set forth above. One is whether adolescent fluoxetine treatment exerts lasting effects in IMS Balb/c mice and delays (or prevents) the progression of the IMS phenotype during aging. The second is whether adult fluoxetine treatment, like adolescent fluoxetine treatment, exerts stimulatory effects on histone expression and enhanced antidepressant efficacy when HDAC activity is reduced. The third question is whether the onset of antidepressant effects of fluoxetine in adulthood is faster in the presence of an HDAC inhibitor. At present, much research focuses on identifying specific genes as targets for the treatment of mood disorders. However, it is possibly more effective to target the levels of histone expression and their posttranslational modifications in subjects with a history of early life stress. It is expected that patients who respond to fluoxetine and those who do not differ in their epigenetic phenotypes. Thus, the results from these studies would indicate the clinical efficacy of HDACi/SSRI co-treatment in patients with low responsiveness to SSRI.

### Example 15

### Fluoxetine's Stimulation of Histone H4 Expression is Dependent Upon Enhanced Serotonergic Signaling and it is a Critical Determinant of Antidepressant Efficacy

The following studies will be performed on Balb/c mice exposed to a powerful paradigm of early life stress in rodents, infant maternal separation (i.e., a daily 3 hour separation of pups from their mothers, starting at P2 and ending at P15). Standard-facility-reared Balb/c mice will serve as controls.

What is the Role of 5-HT in Mediatinq the Effect of Fluoxetine on Histone H4 Expression in Mice with Reduced HDAC Activity?

To address this question, studies will be conducted on IMS Balb/c mice that have reduced HDAC activity (Table 3). They will either be treated with tianeptine (10 mg/kg/day in drinking water) or 5-HT-depleted while treated with fluoxetine (16 mg/kg/day in drinking water) between postnatal ages P35 and P59. IMS mice treated only with fluoxetine or only with depleted 5-HT will serve as controls. With these treatments, the role of elevated 5-HT levels in mediating the effects of antidepressant drugs on histone expression will be directly tested. In contrast to fluoxetine, tianeptine does not significantly alter extracellular 5-HT levels (Mennini et al., 1987) and, in 5-HT-depleted IMS mice, fluoxetine treatment cannot lead to elevated levels of 5-HT. Hence, if elevated levels of 5-HT are indeed critical in mediating the effects of antidepressant drugs on histone expression in mice with reduced HDAC activity, neither tianeptine nor fluoxetine administered to 5-HT-depleted mice should alter histone expression. To test this, the expression of the histones listed in Table 3 will be measured and compared using Western blotting of forebrain neocortical protein. Additionally, the behavioral effects of the treatments will be monitored using the EPM and FST tests, as well as other cognitive test battery as set forth above. Hence, while the role of 5-HT in stimulating histone expression will be examined, the antidepressant efficacies of the two treatments and their effects on cognitive functioning will also be compared to that of fluoxetine alone.

If these studies confirm, as expected, a 5-HT-dependent mechanism underlying the fluoxetine-induced increase in histone H4 expression, additional studies that examine the role of 5-HT receptor-mediated signaling will be performed. The 5-HT1A receptor will be focused on especially because, in the visual system, the mechanisms by which fluoxetine promotes the recovery of sensory function after long-term deprivation in adulthood (epigenetic remodeling via increased expression of aCH3K9 and decreased expression of HDAC5) is completely blocked by WAY-100635, a selective 5-HT1A-receptor antagonist (Vetencourt et al., 2011, Gurevich et al., 2002). This finding showed that the fluoxetine-induced re-instatement of plasticity in adulthood requires signaling through 5-HT1A receptors, and it is very likely that the same 5-HT1A-receptor-mediated mechanism also operates in this model. To test this, the mPFC of fluoxetine-treated IMS Balb/c mice will be infused with WAY-100635 (10 μM; delivered via mini-osmotic pumps at a rate of 0.5 ml/hour during fluoxetine treatment). At the end of the treatment, the behavior in the EPM and FST as well as in the cognitive test battery will be examined, and the expression of histones will be measured as described above. For comparison, these studies will also test the effects of other 5-HT-receptor antagonists, including antagonists of 5-HT2 receptors.

Based on prior results of similar studies, it is expected that a minimal treatment group size of 6 animals for Western blotting and 8 for the behavioral studies. To achieve effective 5-HT depletion in IMS mice, the mice will be treated with 300 mg/kg para-chlorophenylalanine (pCPA; an irreversible inhibitor of tryptophane hydroxylase) twice per day. This treatment leads to a about 80% reduction of 5-HT/5-HIAA levels in the forebrain neocortex within 5 days without affecting the overall well-being of the animal, but with notable effects on 5-HT-regulated processes (Gurevich et al., 2002). The 5-HT/5-HIAA levels of pCPA-treated mice will be monitored by HPLC (Id.). HPLC measures will be taken in 2-day intervals, and the dose of pCPA will be adjusted such that mice never experience more than an 80% of 5-HT depletion.

Does an HDAC Inhibitor Alone Exert Antidepressant Effects in Mice with Normal HDAC Activity but Elevated Extracellular Levels of 5-HT?

To address this question, knockout mice lacking expression of the serotonin transporter (SERT) will be used. These mice are commercially available from Jackson Laboratories. These mice have 10 times higher levels of extracellular 5-HT in the medial prefrontal cortex (mPFC) (Shen et al., 2004, Kim et al., 2005). They are behaviorally insensitive to fluoxetine treatment, and show increased anxiety in the EPM and light/dark exploration test (Holmes et al., 2002, Holmes et al., 2003). Thus, in a first experiment, baseline levels of HDACs and histones between wild type and SERT KO mice will be compared. Then, these mice will be treated from P35 to P59 with the class I/II HDAC inhibitor SAHA (200 mg/kg/day in drinking water) or the class I HDAC inhibitor MS-275 (15 μM/kg/day, a dose that selectively inhibits HDACs 1 and 3; see Dulawa et al., 2004). At the end of these treatments, histone expression will be compared, and behavior in the EPM and FST will be measured. If elevated extracellular levels of 5-HT are sufficient to allow HDAC inhibitors (HDACis) to stimulate histone expression, HDACi-treated SERT KO mice should exhibit increased histone expression. If so, it will be uncovered whether this effect alone is sufficient to elicit antidepressant effects, in particular because SERT KO mice do not normally respond to fluoxetine. Hence, a strong case could be made for a mechanism by which reduced HDAC activity enables an SSRI to stimulate histone expression that, in turn, enhances the antidepressant efficacy.

### Can Fluoxetine Treatment of Adolescent IMS Balb/c Mice Delay the Onset of Histone Depletion and Cognitive Deficits During Aging?

Adolescent fluoxetine treatment of IMS Balb/c mice leads to increased expression of total histone H3 and H4 in young adults (2 months; see, e.g., Table 7). Whether this effect has lasting consequences for the extent of histone depletion during aging will be tested. In these experiments, IMS mice will be treated with fluoxetine (16 mg/kg/day) contained in drinking water between P35 and P59 (adolescence). Control IMS mice receive regular drinking water. Histone expression and telomere lengths will be monitored in the mPFC and hippocampus of non-treated and fluoxetine-treated IMS mice at 2, 3, 5, 8 and 12 months of age, and the behavioral effects of this treatment will be examined in the EPM and FST as well as in the cognitive test battery at these ages. These experiments will include an additional group of IMS mice that were also exposed to the chronic ultra-light stress (CUMS) paradigm in adulthood. Specifically, histone expression, emotive and cognitive behaviors of IMS-CUMS mice that were treated with fluoxetine during adolescence and then exposed to the 1-week CUMS paradigm at 2, 3, and 4 months of age will be examined.

Does Adolescent and Adult Fluoxetine Treatment Exert the Same Effect on Histone Expression and, if so, does HDACi Co-Treatment Lead to a Faster Onset of Antidepressant Effects of Fluoxetine?

There is also evidence for adaptive changes of histone modifications and HDAC expression after chronic adult stress that exert effects that are similar to the effects of antidepressant drug treatment (Uchida et al., 2011, Tsankova et al., 2006, Wilkinson et al., 2009, Renthal et al., 2007, Covington et al., 2009). However, it is not known whether adult antidepressant treatment also stimulates histone expression when HDAC activity is reduced, and whether this also enhances the antidepressant efficacy. To test this, adult IMS Balb/c mice will be treated with fluoxetine for 4 weeks (between P60 and P88; i.e., the average time normally required for fluoxetine to begin to exert antidepressant effects in humans). Non-treated IMS mice will serve as controls. In parallel, adult SFR mice will be co-treated with fluoxetine and one of the two HDACis described above (SAHA or MS-275), and non-treated and fluoxetine-only treated non-stressed mice serve as controls. At the end of these treatments, histone expression will be measured, and the behavior in the EPM and FST as well as in the cognitive test battery will be monitored as described above.

If, as expected, adult fluoxetine treatment of IMS mice and the HDACi/fluoxetine co-treatment of SFR mice indeed enhance the antidepressant effects of fluoxetine and stimulate histone expression after a 28-day treatment, whether the onset of antidepressant effects is faster compared to fluoxetine-only treated mice will be tested. Hence, the treatments (including the fluoxetine-only treatment) will be terminated after 7, 14, and 21 days. The emotive and cognitive behaviors as well as histone expression will be assessed. Additionally, whether adult fluoxetine treatment of IMS Balb/c mice (P60 to P88) will also delay the onset of their histone depletion during aging will be tested.

It is expected that the studies set forth in the Examples above will demonstrate the roles that reduced HDAC activity and elevated 5-HT-mediated signaling play in stimulating histone expression, and how this relates to the antidepressant efficacy.

In sum, the inventor has identified for the first time persistent changes in histone modifications that result from early life stress and that influence emotive behavior and antidepressant treatment response. Balb/c mice exposed to early life stress develop epigenetic modifications that are further augmented by antidepressant treatment with fluoxetine during adolescence. These findings support that, in animal models of chronic stress, HDAC inhibitors have antidepressant effects. They also indicate that subjects with low responsiveness to antidepressant drugs could benefit from a combined treatment with HDAC inhibitors. Importantly, this type of combined treatment—initiated during adolescence—could be the most effective treatment for psychiatric patients with a history of early life stress.

The strategy to use both stress-susceptible and stress-resilient mouse strains to disentangle adaptive from maladaptive molecular responses to ELS exposure allowed for the identification for the first time, of stable, and highly specific epigenetic markers of ELS exposure, factors that directly influence gene expression and behavior as well as antidepressant treatment response. These findings break completely new ground on the role of epigenetic mechanisms in mediating the lasting effects of ELS. Specifically, a novel animal model is now available for the study of the epigenetic influence on cognitive functions governed by the prefrontal cortex (PFC). Moreover, the results indicate that the epigenetic markers of ELS Balb/c mice influence the efficacy of antidepressant drugs by means of stimulating histone expression. Finally, the finding that, without intervention, ELS accelerates histone depletion during aging and thereby, leads to a late-onset progression of cognitive deficits (a process that could possibly also be reversed by an antidepressant treatment that stimulates histone expression) is completely novel.

### Example 16

### Further Studies Linking HDAC Activity, Elevated 5-HT Levels, and Antidepressant Efficacy

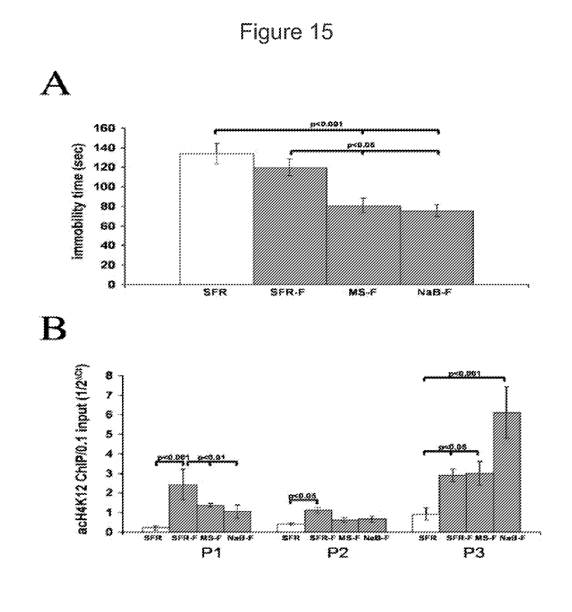

Our data shows that reduced HDAC activity enhances the efficacy of SSRIs and other drugs that increase extracellular 5-HT levels. To further corroborate the link between HDAC activity, elevated 5-HT levels, and antidepressant efficacy, a mouse model of stably elevated 5-HT levels and no response to antidepressant drugs may be used to test whether HDAC inhibitors (HDACis) alone exert antidepressant effects and increase histone H4 expression. Moreover, although it was found that IMS Balb/c mice exhibit reduced expression of class I HDACs 1, 3, and 8 and class II HDACs 7 and 10, two additional findings suggest that the hyperacetylated histone H4 phenotype of IMS Balb/c mice is largely accounted for by reduced class I HDAC activity, especially HDACs 1 and 3. First, adolescent treatment with theophylline at a dose that activates only HDACs 1 and 3 (Ito et al., 2002) reversed the increased expression of aCH4K12 in IMS mice. Second, treatment of SFR mice with the class I-selective HDAC inhibitor MS-275 (also named entinostat) (Simonini et al., 2006) between P35 to P59 resulted in increased aCH4K12/K8/K5 expression (FIG. 14). Hence, the data suggest that class I HDACs play a major role in establishing the histone H4 hyperacetylation phenotype in IMS mice. As further discussed below, this is in contrast to findings of antidepressant effects of reduced class II HDAC activity in adulthood.

To investigate the effects of HDACis in mice with normal HDAC activity and elevated extracellular 5-HT levels, knockout mice lacking expression of the serotonin transporter (SERT; available from Jackson Laboratories) that have 10 times higher levels of extracellular 5-HT in the Medial Prefrontal Cortex (mPFC) (Shen et al., 2004; Kim et al., 2005; Li et al., 2000) will be used. These mice are behaviorally insensitive to fluoxetine treatment, and show increased anxiety in the EPM and light/dark exploration test (Holmes et al., 2002; Holmes et al, 2003). Thus, they are a highly suitable for testing whether HDACis alone are capable of exerting antidepressant effects when 5-HT levels are elevated. In our experiments, baseline levels of forebrain neocortical HDAC mRNA and histone proteins between wild type (WT) and SERT KO mice will be compared. Then, both groups of mice will be administered from P35 to P59 with the broader acting class I/II HDACi SAHA (200 mg/kg/day in drinking water) (Butler et al., 2000; Hockly et al., 2003) or, for direct comparison, the class I-selective HDACi MS-275 (15 μM/kg/day in drinking water, a dose that selectively inhibits HDACs 1 and 3 in the frontal cortex (Simonini et al., 2006)). At the end of these treatments, behavior (in the EPM and FST) will be examined, and histone expression will be compared as described above. If elevated extracellular levels of 5-HT are sufficient to enable HDACis to stimulate histone H4 expression, HDACi-treated SERT KO mice should exhibit increased histone H4 expression. Moreover, if increased histone H4 expression is a critical determinant of antidepressant efficacy, HDACi-treated SERT KO mice should exhibit an improved emotional phenotype in the FST and EPM. Moreover, if MS-275 exerts the anticipated effect, the role of class I HDACs will be further corroborated with studies on fluoxetine-treated IMS mice co-treated with the HDAC1/3-activating dose of theophylline, which should block the effect of fluoxetine.

Whether adult fluoxetine/HDACi co-treatment exerts the same effect on histone expression and whether such co-treatment speeds the onset of antidepressant effects may also be further investigated. In addition to the finding of an epigenetic response to early life stress, adaptive changes in histone modifications and HDAC expression after chronic adult stress have been reported to have antidepressant-like effects (Tsankova et al., 2006; Wilkinson et al., 2009; Covington et al., 2011; Renthal et al., 2007; Covington et al., 2009; Krishnan et al., 2007). However, the HDACs implicated (HDACs 5 and 9) are class II HDACs, and the histone affected is histone H3. Moreover, whether adult antidepressant treatment also stimulates histone expression (either H3 or H4; note the results shown in Table 7) when HDAC activity is reduced, and whether this is accompanied with increased antidepressant efficacy, has not been investigated. To test this, IMS Balb/c mice will be treated with fluoxetine for 4 weeks (between P60 and P88; i.e., the average time normally required for fluoxetine to elicit antidepressant effects in humans). Non-treated IMS mice serve as controls. In parallel, adult SFR mice will be co-treated with fluoxetine and either the class I HDACi MS-275 or the class I/II HDACi SAHA as described above (non-treated and fluoxetine-only treated SFR mice serve as controls). At the end of these treatments, the behavior in the EPM and FST will be monitored, and histone expression will be measured as described above. In addition, if adult fluoxetine treatment of IMS mice and the HDACi/fluoxetine co-treatment of SFR mice indeed enhance the antidepressant effects of fluoxetine and stimulate histone expression after a 28-day treatment, whether the onset of antidepressant effects is faster compared to fluoxetine-only treated mice will be examined. Hence, the treatments will be terminated after 7, 14, and 21 days, and the emotional behavior as well as histone expression will be assessed.

The studies on IMS Balb/c mice recapitulated several findings from human subjects with depression, including increased 5-HT2C-receptor editing (Bhansali et al., 2007; Schmauss et al., 2010) and decreased glucocorticoid receptor expression in the frontal cortex (Navailles et al., 2010), increased stress reactivity, and cognitive deficits in adulthood (Mehta and Schmauss, 2011). These results underscore the biological relevance of this strain to human stress-related disorders. Here, a mechanistic link between increased antidepressant response to adolescent fluoxetine, decreased HDAC activity, and increased acetylation of histone H4 protein in the forebrain neocortex, i.e., an anatomic region encompassing the entire PFC that exerts “top down” control over emotional behavior (Bishop et al., 2004) has been shown. Several mouse models that are highly suited for demonstrating the roles of reduced HDAC activity and increased 5-HT-triggered signaling in stimulating histone expression and enhancing antidepressant efficacy will be used.

Whether changing levels of histone acetylation in peripheral blood during antidepressant treatment parallel the changing levels found in the frontal cortex will also be tested. To test this, trunk blood will be collected at the time of sacrifice and lymphocytes will be isolated by Ficoll-gradient centrifugation. Histone acetylation in frontal cortex and lymphocytes will then be measured with Western blots. If the studies on lymphocytes are successful, then a peripheral biomarker for predicting antidepressant efficacy will be provided.

It is expected that adult fluoxetine treatment, like adolescent fluoxetine treatment, exerts stimulatory effects on histone expression and enhances antidepressant efficacy (with possibly earlier onset) when HDAC activity is reduced.

### Example 17

### Clinical Study on Patients with Major Depression

A clinical study on patients with major depression that do and that do not respond to SSRI treatment will be conducted. It would be relatively straightforward to compare their histone H4 acetylation levels in peripheral blood (lymphocytes), as such measures have already proven to yield informative data for patients treated with VPA (Sharma et al., 2006). This would directly speak to the role of the epigenetic phenotype in influencing the antidepressant efficacy of SSRIs. Further, these patients may also be treated with both HDAC inhibitors and SSRIs. It is expected that HDACi/SSRI co-treatment in patients with low responsiveness to SSRIs will have clinical efficacy.

### DOCUMENTS

Afifi T O, Enns M W, Cox B J, Asmundson G J G, Stein M B, Sareen J. Population attributable fractions of psychiatric disorders and suicide ideation and attempts associated with adverse childhood experiences. Am J Public Health 2008; 98: 946-52.
Aggleton J P, Neave N, Nagle S, Sahgal A (1995) A comparison of the effects of medial prefrontal, cingulate cortex, and cingulum bundle lesions on tests of spatial memory: Evidence of a double dissociation between frontal and cingulum bundle contributions. J Neurosci 15: 7270-7281.
Archer B T, Özçelik T, Jahn R, Francke U, Südhof T C (1990) Structures and chromosomal localization of two human genes encoding synaptobrevins 1 and 2. J Biol Chem 265: 17267-17273.
Barrett R M, Wood M A (2008) Beyond transcription factor: The role of chromatin modifying enzymes in regulating transcription required for memory. Learn Mem 15: 460-467.
Bhansali P, Dunning J, Singer S E, David L, Schmauss C (2007) Early life stress alters adult serotonin 2C receptor pre-mRNA editing and expression of the alpha subunit of heterotrimeric G protein Gq. J Neurosci 27: 1467-1473. PMID: 17287521 (Free Article)
Bhansali P, Dunning J, Singer S E, David L, Schmauss C. Early life stress alters adult serotonin 2C receptor pre-mRNA editing and expression of the alpha subunit of heterotrimeric G protein Gq. J Neurosci 2007; 27: 1467-73.
Bishop S, Duncan J, Brett M, Lawrence A D (2004) Prefrontal cortical function and anxiety: Controlling attention to threat-related stimuli. Nat Neurosci 7: 184-188.
Birrell J M, Brown V J (2000) Medial frontal cortex mediates perceptual attention set shifting in the rat. J Neurosci 20: 4320-4324.
Boss E B, Janssen W G, Ohm D T, Yuk F J, Wadsworth S, Saardi K, McEwen B S, Morrison J H (2011) Evidence for reduced experience-dependent dendritic spine plasticity in the aging prefrontal cortex. J Neurosci 31: 7831-7839.
Braun K, Lange E, Metzger M, Poeggel G (2000) Maternal separation followed by early social deprivation affects the development of monoaminergic fiber systems in the medial prefrontal cortex of octdon degus. Neurosci 95: 309-318.
Brown M W, Aggleton J P (2001) Recognition memory: What are the roles of the perirhinal cortex and the hippocampus? Nat Rev Neurosci 2: 51-61.
Brunson K L, Kramar E, Lin B, Colgin L L, Yanagihara T K, Lynch G, Baram T Z (2005) Mechanisms of late-onset cognitive decline after early life stress. J Neurosci 25: 9328-9338.
Butler L M, Agus D B, Scher H I, Higgins B, Rose A, Cordon-Cardo C, Thaler H T, Rifkind R A, Marks P A, Richon V M (2000) Suberoylanidide hydroxamic acid, an inhibitor of histone deacetylase, suppresses the growth of prostate cancer cells in vitro and in vivo. Cancer Res 60: 5165-5170.
Caspi A, Hariri A, Holmes A, Uher R, Moffitt T E (2010) Genetic sensitivity to the environment: The case of the serotonin transporter gene and its implications for studying complex diseases and traits. Am J Psychiat 167: 509-527.
Covington III H E, Maze I, LaPlant Q C, Vialou V F, Ohnishi Y N, Berton O, Fass D M, Renthal W, Rush III A J, Wu E Y, Ghose S, Krishnan V, Russo S J, Tamminga C, Haggarty S J, Nestler E J (2009) Antidepressant actions of histone deacetylase inhibitors. J Neurosci 29: 11451-11460.
Covington H E, Maze I, Sun H, Bomze H M, DeMaio K D, Wu E Y, Dietz D M, Lobo M K, Ghose S, Mouzon E, Neve R L, Tamminga C A, Nestler E J (2011) A role for repressive histone methylation in cocaine induced vulnerability to stress. Neuron 71: 656-670.
Davis S, Renaudineau S, Poirier R, Poucet B, Save E, Laroche S (2010) The formation and stability of recognition memory: what happens upon recall? Frontiers Beh Neurosci 4: Article 177. doi:10.3389/fnbeh.2010.00177
Day J J, Sweatt J D (2011). Epigenetic mechanisms in cognition. Neuron 70: 813-829.
de Ruijter A J M, van Gennip A H, Caron H N, Kemp S, van Kuilenburg A B P. Histone deacetylases (HDACs): characterization of the classical HDAC family. Biochem J 2003; 370: 737-749.
DeSteno D A, Schmauss C (2008) Induction of early growth response gene 2 expression in the forebrain of mice performing an attention-set-shifting task. Neurosci 152: 417-428. PMCID: PMC2323625.
Dulac C. Brain function and chromatin plasticity. Nature 2010; 465: 728-35.
Dulawa S, Holick K A, Gundersen B, Hen R (2004) Effects of chronic fluoxetine in animal models of anxiety and depression. Neuropsychopharmacol 29:1321-1330.
Englander M T, Dulawa S, Bhansali P, Schmauss C (2005) How stress and fluoxetine modulate serotonin 2C receptor pre-mRNA editing. J Neurosci 25: 648-651. PMID: 15659601 (Free Article)
Erlenmeyer-Kimling L, Rock D, Roberts S A, Jamal M, Kestenbaum C, Cornblatt B, Adamo U H, Gottesman I I (2000) Attention, memory, and motor skills as childhood predictors of schizophrenia-related psychosis: The New York High Risk Project. Am J Psychiat 157: 1416-1422.
Feser J, Truong D, Das, C, Carson J J, Kieft J, Harkness T, Tyler J K (2010) Elevated histone expression promotes life span extension. Mol Cell 39: 724-735.
Feser J, Tyler J (2011) Chromatin structure as a mediator of aging. FEBS Lett (2011), doi: 10.1016/febslet.2010.11.016
Fischer A, Sananbebesi F, Mungenast A, Tsai L-H. Targeting the correct HDAC(s) to treat cognitive disorders. Trends Pharmacol Sci 2010; 31: 605-17.
Flanary B E, Streit W J (2003) Telomere shorten with age in rat cerebellum and cortex in vivo. J Anti Aging Med 6: 299-308.
Flanary B E, Streit W J (2005) Efects of axotomy on telomere length, telomerase activity, and protein in activated microglia. J Neurosci Res 82: 160-171.
Fournier J C, DeRubeis R J, Holton S D, Dimidjian S, Amsterdam J D, Shelton R C, Fawcett J (2010) Antidepressant drug effects in depression severity. A patient-level meta-analysis. JAMA 6: 47-53.29.
Francis D D, Szegda K, Campell G, Martin W D, Insel T R (2003) Epigenetic sources of behavioral differences in mice. Nat Neurosci 6: 445-446.
Glickstein S B, DeSteno D A, Hof P R, Schmauss C (2005) Mice lacking dopamine D₂ and D₃ receptors exhibit differential activation of prefrontal cortical neurons during tasks requiring attention. Cer Cor 15: 1016-1024. PMID: 15537671
Glickstein S B, Hof P R, Schmauss C (2002) Mice lacking dopamine D2 and D3 receptors have spatial working memory deficits. J Neurosci 22: 5619-5629. PMID: 12097513 (Free Article)
Gottesman I I, Erlenmeyer-Kimling L (2001) Family and twin strategies as a head start in defining prodromes and endophenotypes for hypothetical early interventions in schizophrenia. Schizophrenia Res 51: 93-102.
Gottlicher M, Minucci S, Zhu P, Kramer O H, Schimpf A, Giavara S, Sleeman J P, Lo Coco F, Nervi C, Pelicci P G, Neinzel T (2001) Valproic acid defines a novel class of HDAC inhibitors inducing differentiation of transformed cells. EMBO J. 20: 6969-6978.
Green J G, McLaughlin K A, Berglund P A, Gruber M J, Sampson N A, Zaslaysky A M, Kessler R C (2010) Childhood adversities and adult psychiatric disorders in the National Comorbidity Survey Replication I: associations with first onset of DSM-IV disorders. Arch Gen Psychiatry 67: 113-123.
Guan J-S, Haggarty S J, Giacometti E, Dannenberg J-H, Joseph N, Gao J, Nieland T J F, Zhou Y, Wang X. Mazitschek R, Bradner J E, DePinho R A, Jaenisch R, Tsai L-H (2009), HDAC2 negatively regulates memory formation and synaptic plasticity. Nature 459: 55-60.
Gurevich I, Englander M T, Adlersberg M, Siegal N B, Schmauss C (2002) Modulation of serotonin 2C receptor editing by sustained changes in serotonergic neurotransmission. J Neurosci 22: 10529-10932. PMID: 12486144 (Free Article).
Haberland M, Montgomery R L, Olson E N (2009) The many roles of histone deactylases in development and physiology: Implications for disease and therapy. Nat Rev Gen 10: 32-42.
Hara Y, Park S, Janssen W G, Punsoni M, Rapp P R, Morrison J H (2011) Synaptic characteristics of dentate gyrus axonal boutons with aging, menopause, and memory in female rhesus monkeys. J Neurosci 31: 7737-7744.
Helmeke C, Ovtscharoff J R W, Poeggel G, Braun K (2008) Imbalance of immunohistochemically characterized interneuron populations in the adolescent and adult rodent medial prefrontal cortex after repeated exposure to neonatal separation stress. Neurosci 152: 18-28.
Hockly E, Richon V M, Woodman B, Smith D L, Zhou X, Rosa E et al. Suberoylanilide hydroxamic acid, a histone deacetylase inhibitor, ameliorates motor deficits in a mouse model of Huntington's disease. Proc Natl Acad Sci USA 2003; 100: 2041-46.
Holmes A, le Guisquet A M, Vogel E, Millstein R A, Leman S, Belzung C (2005), Early life genetic, epigenetic, and environmental factors shaping emotionality in rodents. Neurosci Biobehav Rev 29: 1335-1346.
Holmes A, Li Q, Murphy D L, Gold E, Crawley J N (2003) Abnormal anxiety-related behavior in serotonin transporter null mutant mice: the influence of the genetic background. Genes Brain Behav 2: 365-380.
Holmes A, Yang R J, Murphy D L, Crawley J N (2002) Evaluation of antidepressant-related behavioral responses in mice lacking the serotonin transporter. Neuropsychopharmacol 27: 914-923.
Ito K, Lim K, Caramori G, Cosio B, Chung F, Adcock I M, Barnes P J (2002) A molecular mechanism of action of theophylline: Induction of histone deacetylase activity to decrease inflammatory gene expression. Proc Natl Acad Sci USA 99: 8921-8926.
Kilgore M, Miller C A, Fass D M, Hennig K M, Haggarty S J, Sweeatt J D, Rumbaugh D (2010) Inhibitors of class I histone deacetylases reverse contextual memory deficits in a mouse models of Alzheimer's disease. Neuropsychopharmacol 35: 870-880.
Kim D-K, Tolliver T J, Huang S-J, Martin B J, Andrews A M, Wicherms C, Holmes A, Lesch K-P, Murphey D L (2005) Altered serotonin synthesis, turnover and dynamic regulation in multiple brain regions of mice lacking the serotonin transporter. Neuropharmacol 49: 798-810.
Kolber B J, Boyle M P, Wieczorek L, Kelley C I, Onwuzurike C C, Nettles S A, Vogt S K, Muglia L J (2010) Transient early-life forebrain corticotropin-releasing hormone elevation causes long-lasting anxiogenic and despair-like changes in mice. J Neurosci 30: 2571-2581.
Korosi A, Shanabrough M, McClelland S, Liu Z-W, Borok E, Gao X-B, Horvath T L, Baram T Z (2010) Early-life experience reduces excitation to stress-responsive hypothalamic neurons and reprograms the expression of corticotropin-releasing hormone. J Neurosci 30: 703-713.
Krishnan V, Han M-H, Graham D L, Berton O, Rehthal W, Russo S J, LaPlant Q, Graham A, Lutter M, Lagace D C, Ghose S, Reister R, Tannous P, Green T A, Neve R L, Chakravarty S, Kumar A, Eisch A J, Self, D, Lee F S, Tamminga C A, Cooper D, Gershenfeld H K, Nestler E J (2007) Molecular adaptations underlying susceptibility and resistance to social defeat in brain reward regions. Cell 131: 391-404.
Kuozarides T (2007) Chromatin Modifications and their Function. Cell 128: 693-705.
Kwan T, Benovoy D, Dias C, Gurd S, Serre D, Zuzan H, Clark et al. Heritability of alternative splicing in the human genome. Genome Res 2007; 17: 1210-18.
Ladd C O, Huot R L, Thrivikraman K V, Nemeroff C B, Plotsky P M (2003) Long-term adaptation in glucocorticoid receptor and mineralocorticoid receptor mRNA and negative feedback on the hypothalamo-pituitary-adrenal axis following neonatal maternal separation. Biol Psychiat 55: 367-375.
Lanfumey L, Pardon M C, Laaris N, Joubert C, Hanoun N, Hamon M, Cohen-Salmon C (1999) 5-HT1A autoreceptor desensitization by chronic ultramild stress in mice. NeuroReport 10: 3369-3374.
Lee H, Rezai-Zadeh N, Seto E. Negative regulation of histone deacetylase 8 activity by cyclic AMP-dependent protein kinase A. Mol Cell Biol 2004; 24: 765-73.
Levine A A, Guan Z, Barco A, Xu S, Kandel E R, Schwartz J H. CREB-binding protein controls response to cocaine by acetylating histones at the fosB promoter in the mouse striatum. Proc Natl Acad Sci USA 2005; 102: 19186-91.
Levine, A., Worrell, T. R., Zimnisky, R. & Schmauss, C. Early life stress triggers sustained changes in histone deacetylase expression and histone H4 modifications that alter responsiveness to adolescent antidepressant treatment. Neurobiol. Dis. 45, 488-498 (2012).
Levine S (1994) The ontogeny of the hypothalamic-pituitary-adrenal axis. The influence of maternal factors. Ann NY Acad Sci 746: 275-288.
Li Q, Wichems C, Heils A, Lesch K-P, Murphey D L (2000) Reduction in the density and expression, but not G-protein coupling, of serotonin receptors (5-HT_{1A}) in 5-HT transporter knockout mice: Gender and brain region differences. J Neurosci 20: 7888-7895.
Li Y, Camp S, Rachinsky T L, Bongiorno C, Taylor P (1993) Promotor elements and transcriptional control of the mouse acetylcholinesterase gene. J Biol Chem 268: 3563-3572.
Lukas M, Bredewold R, Neuman I D, Veenema A H (2010) Maternal separation interferes with developmental changes in brain vasopressin and oxytocin receptor binding in male rats. Neuropharmacol 58: 78-87.
Marazziti D, Consoli G, Picchetti M, Carlini M, Faravelli L (2010) Cognitive impairment in major depression. Eur J Pharmacol 626: 83-86.
McAlonan K, Brown V J (2003) Orbital frontal cortex mediates reversal learning and not attention set shifting in the rat. Beh Brain Res 146: 97-103.
McEwen B S, Chattarji S, Diamond D M, Jay T M, Reagan L P, Svenningsson P, Fuchs E (2010) The neurobiological properties of tianaptine (Stablon): from monoamine hypothesis to glutamatergic modulation. Mol Psychiat 15: 237-249.
McQuown S C, Barrett R M, Matheos D P, Post R J, Rogge G A, Alenghat T, Mullican S E, Jones S, Rusche J R, Lazar M A, Wood M A (2011) HDAC3 is a negative regulator of long-term memory formation. Neurosci 31: 764-774.
Mehta M, Schmauss C. Strain-specific cognitive deficits in mice exposed to early life stress. Beh Neurosci 2011; 125: 29-36.
Mennini T, Mocaer E, Garattini S (1987) Tianeptine, a selective enhancer of serotonin uptake in rat brain. NS Arch Pharmacol 336: 478-482.
Millstein R A, Holmes A (2007) Effects of repeated maternal separation on anxiety- and depression-related phenotypes in different mouse strains. Neurosci Beh Rev 31: 3-17.
Mizoguchi K, Ishige A, Aburada M, Tabira T (2003) Chronic stress attenuates glucocorticoid negative feedback: involvement of the prefrontal cortex and hippocampus. Neurosci 119: 887-897.
Moriceau S, Shinoya K, Sullivan R M (2009) Early-life stress disrupts attachment learning: The role of amygdala cortocosterone, locus ceruleus corticotropin release hormone, and olfactory bulb norepinephrine. J Neurosci 29: 15745-15755.
Mozhui K, Karlsson R-M, Kash T L, Ihne J, Norcross M, Patel S, Farrell M R, Hill E E, Graybeal C, Martin K P, Camp M, Fitzgerald P J, Ciobanu D C, Sprengel R, Mishina M, Wellman C L, Winder D G, Williams R W, Holmes A (2010) Strain differences in stress responsivity are associated with divergent amygdala gene expression and glutamate-mediated neuronal excitability. J Neurosci 30: 5357-5367.
Murgatroyd C, Patchev A, Wu Y, Micale V, Bockmühl Y, Fischer D et al. Dynamic DNA methylation programs persistent adverse effects of early-life stress. Nat Neurosci 2009; 12: 1559-66.
Navailles S, Hof P R, Schmauss C (2008) Antidepressant stimulation of hippocampal neurogenesis is age-dependent and affected by early life stress. J Comp Neurol 509: 372-381. PMCID: PMC2502065
Navailles S, Zimnisky R, Schmauss C (2010) Expression of glucocorticoid receptor and early growth response gene 1 during postnatal development of two inbred strains of mice exposed to early life stress. Dev Neurosci 32: 139-148. PMCID—In Process
Nemeroff C B, Heim C M, Thase M E, Klein D N, Rush A J, Schatzberg A F, Ninan P T, McCullough J P Jr, Weiss P M, Dunner D L, Rothbaum B O, Kornstein S, Keitner G, Keller M B (2003) Differential responses to psychotherapy versus pharmacotherapy in patients with chronic forms of major depression and childhood trauma. Proc Natl Acad Sci USA 100: 14293-14296.
Nikam S S, Tennekoon G I, Christy B A, Yoshino J E, Rutkowski J L (1995) The zinc finger transcription factor zif268/Egr-1 is essential for Schwann cell expression of the p75 NGF receptor. Mol Cell Neurosci 6: 337-348.
Nuechterlein K H, Barch D M, Gold J M, Goldberg J M, Green M F, Heaton R K (2004) Identification of separable cognitive factors in schizophrenia. Schizophrenia Res 15: 29-39.
O'Donovan K J, Tourtellotte W G, Milbrandt J, Baraban J M (1999) The EGR family of transcription-regulatory factors: progress at the interface of molecular and systems neuroscience. Trends Neurosci 22: 167-173.
O'Sullivan R J, Kubicek S, Schreiber S L, Karlseder J (2010) Reduced histone biosynthesis and chromatin changes arising from a damage signal at telomeres. Nat Struct Mol Biol 17: 1218-226.
Özçelik T, Lafreniere R G, Archer B T, Johnston P A, Willard H F, Francke U, Sudhof T C (1990) Synaptophysin: Structure of the human gene and assignment to the X chromosome in man and mouse. Am J Hum Genet 47: 551-561.
Park P J (2009) ChIP-seq: advantages and challenges of a maturing technology. Nature Rev Gen 10: 669-680.
Park S, Holzman P S (1992) Schizophrenics show spatial working memory deficits. Arch Gen Psychiat 49: 975-982.
Pascual R, Zamora-Leon S P (2007) Effects of neonatal maternal deprivation and postweaning environmental complexity on dendritic morphology of prefrontal pyramidal neurons in the rat. Acta Neurobiologiae Experimentalis 67: 471-479.
Peleg S, Sananbenesi F, Zovoitis A, Burkhardt S, Bahari-Javan S, Agis-Balboa R C, Cota P, Wittnam J L, Gogol-Doering A, Opitz L, Salinas-Riester G, Dettenhofer A, Kang H, Farinelli L, Chen W, Fischer A (2010), Altered histone acetylation is associated with memory impairment in mice. Science 328: 753-756.
Penner M R, Roth T L, Barnes C A, Sweatt J D (2010) An epigenetic hypothesis of aging-related cognitive dysfunction. Frontiers Aging Neurosci 2: 1-22 (doi: 10.3389/fnagi.2010.00009)
Petersohn D, Schoch S, Brinkmann D R, Thiel G (1995) The human synapsin II gene promotor. J Biol Chem 270: 24361-24369.
Plotzky P M, Meaney M J (1993): Early, postnatal experience alters hypothalamic corticotropin-releasing factor (CRF) mRNA, median eminence CRF content and stress-induced release in adult rats. Mol Brain Res 18: 195-200.
Renthal W, Maze I, Krishnan V, Covington III H E, Xiao G, Kumar A, Russo S J, Graham A, Tsankova N, Kippin T E, Kerstetter K A, Neve R L, Haggarty S J, McKinsey T A, Bassel-Duby R, Olson E N, Nestler E J (2007) Histone deacetylase 5 epigenetically controls behavioral adaptations to chronic emotional stimuli. Neuron 56: 517-529.
Ryan J M, Cristofalo V J (1972) Histone acetylation during aging of human cells in culture. Biochem Biophys Res Com 48: 735-742.
Schmauss C, Zimnisky R, Mehta M, Shapiro L P (2010) The roles of phospholipase C activation and alternative ADAR1 and ADAR2 pre-mRNA splicing in modulating serotonin 2C receptor editing in vivo. RNA 16: 1779-1785. PMCID: PMC2924537
Schmitz C, Hof P R (2000) Recommendations for straightforward and rigorous methods of counting neurons based on a computer simulation approach. J Chem Neuroanat 20:93-114.
Sharma R P, Rosen C, Kartan S, Guidotti A, Costa E, Grayson D R, Chase K (2006) Valproic acid and chromatin remodeling in schizophrenia and bipolar disorders: Preliminary results from a clinical population. Schizophrenia Res 88: 227-231.
Shen H-w, Hagino Y, Kobayashi H, Shinohara-Tanaka K, Ikeda K, Yamamoto H, Yamamoto T, Lesch K-P, Murphey D L, Hall F S, Uhl G R, Sora I (2004) Regional differences in extracellular dopamine and serotonin assessed by in vivo microdialysis in mice lacking dopamine and/or serotonin transporters. Neuropsychopharmacol 29: 1790-1799.
Simonini M V, Camargo L M, Dong E, Maloku E, Veldic M, Costa E, Guidotti A (2006) The benzamide MS-275 is a potent, long lasting brain region-selective inhibitor of histone deacetylases. Proc Natl Acad Sci USA 103: 1587-1592.
Thiel G, Schoch S, Petersohn D (1994) Regulation of synapsin I gene expression by the zinc finger transcription factor zif268/egr-1. J Biol Chem 269: 15294-15301.
Tsankova N M, Berton O, Renthal W, Kumar A, Neve R L, Nestler E J (2006) Sustained hippocampal chromatin regulation in a mouse model of depression and antidepressant action. Nat Neurosci 9: 519-525.
Tunnicliff G (1999) Action of sodium valproate on the central nervous system. J Physiol Pharmacol 50: 347-365.
Uchida S, Hara K, Kobayashi A, Otsuki K, Yamagata H, Hobara T, Suzuki T, Miyata N, Watanabe Y (2011) Epigenetic status of Gdnf in the ventral striatum determines susceptibility and adaptation to daily stressful events. Neuron 69: 359-372.
Ursini G, Bollati V, Fazio L, Porcelli A, Lacovelli L, Catalani A, Sinibaldi L, Gelao B, Romano R, Rampino A, Taurisano P, Mancini M, Di Giorgio A, Popolizio T, Baccarelli A, de Blasi A, Blasi G, Bertolini A (2011) Stress-related methylation of the catechol-O-methyltransferase Val¹⁵⁸ allele predicts human prefrontal cognition and activity. J Neurosci 31: 6692-6698.
Vetencourt J F M, Sale A, Viegi A, Baroncelli L, De Pasquale R, O'Leary O, Castrén E, Maffei L (2008) The antidepressant fluoxetine restores plasticity in the adult visual cortex. Science 320: 385-388.
Vetencourt J F M, Tiraboschi E, Spolidoro M, Castren E, Maffei L (2011) Serotonin triggers a transient epigenetic mechanism that reinstates adult visual cortex plasticity in the rat. Eur J Neurosci 33: 49-57.
Wang Z, Zang C, Cui K, Schones D E, Barski A, Peng W, Zhao K. Genome-wide mapping of HATs and HDACs reveals distinct functions in active and inactive genes. Cell 2009; 138: 1019-31.
West M J, Slomianka L, Gundersen H J G (1991) Unbiased stereological estimation of the total number of neurons in the subdivisions of the rat hippocampus using the optical fractionator. Anat Rec 231: 482-497.
Wilkinson M B, Xiao G, Kumar A, LaPlant Q, Renthal W, Sikder D et al. Imipramine treatment and resiliency exhibit similar chromatin regulation in the mouse nucleus accumbens in depression models. J Neurosci 2009; 29: 7820-32.
Witte E A, Davidson M C, Marocco R T (1997) Effects of altering brain cholinergic activity on covert orienting of attention: comparison of monkey and human performance. Psychopharmacology (Berl) 132: 324-334.
Wyrick J J, Holstege F C P, Jennings E J, Causton H C, Shore D, Grunstein M, Lander E S, Young R A (1999) Chromosomal landscape of nucleosome-dependent gene expression and silencing in yeast. Nature 402: 418-421.
Wysocka J, Reilly P T, Herr W (2001) Loss of HCF-10 chromatin association precedes temperature-induced growth arrest of tsBN67 cells. Mol Cell Biol 21: 3820-3829.
Zhang K, Siino J S, Jones P R, Yau P M, Bradbury E M. A mass-spectrometric Western blot to evaluate the correlation between histone methylation and histone acetylation. Proteomics 2004; 4: 3765-75.
Zhang Z, Pugh B F (2011) High-resolution genome-wide mapping of the primary structure of chromatin. Cell 144: 175-186.
Zihl J, Gron G, Brunnauer A (1998) Cognitive deficits in schizophrenia and affective disorders: evidence for a final common pathway disorder. Acta Psychiat Scan 97: 351-357.

All documents cited in this application are hereby incorporated by reference as if recited in full herein.

Although illustrative embodiments of the present invention have been described herein, it should be understood that the invention is not limited to those described, and that various other changes or modifications may be made by one skilled in the art without departing from the scope or spirit of the invention.

What is claimed is:

1. A method for enhancing the anti-depressant efficacy of a selective serotonin re-uptake inhibitor (SSRI) in a patient being treated for a mood disorder, the method comprising administering to a patient in need thereof a therapeutically effective amount of an SSRI and a therapeutically effective amount of a modulator of histone expression.

2. The method according to claim 1, wherein the modulator of histone expression is a class I histone deacetylase inhibitor (HDACi).

3. The method according to claim 2, wherein the SSRI and HDACi are co-administered.

4. The method according to claim 2, wherein the SSRI and HDACi are administered serially over time.

5. The method according to claim 2, wherein the HDACi is selected from the group consisting of trichostatin A (TSA), suberoylanilide hydroxamic acid (SAHA), MS-275, pyroxamide, azelaic-1-hydroxamate-9-anilide (AAHA), CRA-024781 (Pharmacyclics, Sunnyvale, Calif.), bombesin-2 (BB2) receptor antagonist, JNJ-16241199 (Johnson & Johnson, Langhorne, Pa.), Oxamflatin, CG-1521 (Errant Gene Therapeutics, LLC, Chicago, Ill.), CG-1255 (Errant Gene Therapeutics, LLC, Chicago, Ill.), SK-7068 (In2Gen/SK Chemical Co., Suweon, Korea), SK-7041 (In2Gen/SK Chemical Co., Suweon, Korea), m-carboxycinnamic acid bis-hydroxamide (CBHA), Scriptaid (N-Hydroxy-1,3-dioxo-1H-benz[de]isoquinoline-2(3H)-hexan amide), SB-623 (Merrion Research I Limited, National Digital Park, Ireland), SB-639 (Merrion Research I Limited, National Digital Park, Ireland), SB-624 (Merrion Research I Limited, National Digital Park, Ireland), Panobinostat (LBH-589) (Novartis, Basel, Switzerland), NVP-LAQ824 (Novartis, Basel, Switzerland), butyrate, phenylbutyrate, valporic acid (VPA), Pivanex™ (Titan Pharmaceuticals, Inc.), AN-1 (Titan Pharmaceuticals, Inc.), tributyrin, compound G1, pivaloyloxymethyl butyrate, hyaluronic acid butyric acid ester (HA-But), Apicidine, Trapoxin-A, Trapoxin-B, cyclic hydroxamic acid-containing peptide 1 (CHAP-1), CHAP-31, CHAP-15, chlamidocin, HC-Toxin, WF-27082B (Fujisawa Pharmaceutical Company, Ltd., Osaka, Japan), Romidepsin (Gloucester Pharmaceuticals, Cambridge, Mass.), Spiruchostatin A, Depudesin, compound D1, Triacetylshikimic acid, Cyclostellettamine FFF1, Cyclostellettamine FFF2, Cyclostellettamine FFF3, Cyclostellettamine FFF4, MS-27-275 (Schering AG, Germany), Tacedinaline (N-acetyldinaline), ITF-2357 (Italfarmaco, Cinisello Balsamo, Italy), N-hydroxy-4-(3-methyl-2-phenyl-butyrylamino)benzamide (HDAC-42), MGCD-0103 (MethylGene Inc., Montreal, Quebec, Canada), PX-117794 (TopoTarget AS, København, Denmark), Belinostat (TopoTarget AS, Kobenhavn, Denmark), sulfonamide hydroxamic acid, mocetinostat (MethylGene), Entinostat (MS-275, Bayer AG), MG-2856 (MethylGene), MG-4230 (MethylGene), MG-4915 (MethylGene), MG-5026 (MethylGene), belinostat (TopoTarget AS), abexinostat (Celera), panobinostat (Novartis), CG-200745 (CrystalGenomi), SB-939 (S*BIO), chidamide (HUYA Bioscience), CHR-3996 (Chroma Therapeutics), AR-42 (Arno Therapeutics), RG-2833 (RepliGen), OCID-4681-S-01, (Orchid Pharmaceuticals), PCI-34051 (Pharmalcyclics), DAC-60 (Genextra), KAR-2581 (Karus Therapeutics), resminostat (Nycomed Pharma), 4SC-202 (Nycomed Pharma), YM-753 (Astellas), ACY-1216 (Acetylon), KAR-3000 (Karus Therapeutics), CU-906 (Curis), IKH-02 (IkerChem), ACY-257 (Acetylon), HDAC3 inhibitors (RepliGen), KAR-3166 (Karus Therapeutics), ONCO-101 (Oncoholdings), GSK424887 (GlaxoSmithKline), AGO178 (Novartis), TC5214 (AstraZeneca), pharmaceutically acceptable salts thereof, and combinations thereof.

6. The method according to claim 5, wherein the HDACi is SAHA, VPA, pharmaceutically acceptable salts thereof, or combinations thereof.

7. The method according to claim 1, wherein the SSRI is selected from the group consisting of fluvoxamine, trazodone, indeloxazine, viloxazine, dapoxetine, duloxetin, fluoxetine, olanzapine, tramadol, tramadol, paroxetine mesylate, venlafaxine, citalopram, escitalopram, demexiptiline, vilazodone, nitroxazepine, paroxetine, desvenlafaxine, sertraline, venlafaxine, milnacipran, minaprine, quinupramine, amine transporter inhibitor (AMRI), venlafaxine (Auspex), DSP-1053 (Dainippon Sumitomo Pharma), SEP-228432 (Dainippon Sumitomo Pharma), DA-8031 (DA-8031), escitalopram (Lundbeck), NSD-788 (Neurosearch), SKL-10406 (SK Holdings), BMS-820836 (AMRI), pipamperone, doxepin (Winston Pharmaceuticals), levomilnacipran, clonazepam (Zydus) pharmaceutically acceptable salts thereof, and combinations thereof.

8. The method according to claim 7, wherein the SSRI is fluoxetine, pharmaceutically acceptable salts thereof, or combinations thereof.

9. The method according to claim 1, wherein the patient is a mammal.

10. A method for identifying a patient population that suffers from a mood disorder that is more likely to respond to SSRI treatment comprising:
(a) obtaining a biological sample from the patient;
(b) testing the biological sample to determine whether it has a reduced HDAC activity compared to a control population; and
(c) administering an effective amount of an SSRI to the patient, optionally together with an effective amount HDACi if the biological sample of the patient population has a reduced HDAC activity compared to the control population.

11. The method according to claim 10, wherein the biological sample is selected from the group consisting of body fluid, body tissue, and a portion thereof.

12. The method according to claim 11, wherein the biological sample is peripheral blood lymphocytes.

13. The method according to claim 10, wherein the HDACi is selected from the group consisting of trichostatin A (TSA), suberoylanilide hydroxamic acid (SAHA), MS-275, pyroxamide, azelaic-1-hydroxamate-9-anilide (AAHA), CRA-024781 (Pharmacyclics, Sunnyvale, Calif.), bombesin-2 (BB2) receptor antagonist, JNJ-16241199 (Johnson & Johnson, Langhorne, Pa.), Oxamflatin, CG-1521 (Errant Gene Therapeutics, LLC, Chicago, Ill.), CG-1255 (Errant Gene Therapeutics, LLC, Chicago, Ill.), SK-7068 (In2Gen/SK Chemical Co., Suweon, Korea), SK-7041 (In2Gen/SK Chemical Co., Suweon, Korea), m-carboxycinnamic acid bis-hydroxamide (CBHA), Scriptaid (N-Hydroxy-1,3-dioxo-1H-benz[de]isoquinoline-2(3H)-hexan amide), SB-623 (Merrion Research I Limited, National Digital Park, Ireland), SB-639 (Merrion Research I Limited, National Digital Park, Ireland), SB-624 (Merrion Research I Limited, National Digital Park, Ireland), Panobinostat (LBH-589) (Novartis, Basel, Switzerland), NVP-LAQ824 (Novartis, Basel, Switzerland), butyrate, phenylbutyrate, valporic acid (VPA), Pivanex™ (Titan Pharmaceuticals, Inc.), AN-1 (Titan Pharmaceuticals, Inc.), tributyrin, compound G1, pivaloyloxymethyl butyrate, hyaluronic acid butyric acid ester (HA-But), Apicidine, Trapoxin-A, Trapoxin-B, cyclic hydroxamic acid-containing peptide 1 (CHAP-1), CHAP-31, CHAP-15, chlamidocin, HC-Toxin, WF-27082B (Fujisawa Pharmaceutical Company, Ltd., Osaka, Japan), Romidepsin (Gloucester Pharmaceuticals, Cambridge, Mass.), Spiruchostatin A, Depudesin, compound D1, Triacetylshikimic acid, Cyclostellettamine FFF1, Cyclostellettamine FFF2, Cyclostellettamine FFF3, Cyclostellettamine FFF4, MS-27-275 (Schering AG, Germany), Tacedinaline (N-acetyldinaline), ITF-2357 (Italfarmaco, Cinisello Balsamo, Italy), N-hydroxy-4-(3-methyl-2-phenyl-butyrylamino)benzamide (HDAC-42), MGCD-0103 (MethylGene Inc., Montreal, Quebec, Canada), PX-117794 (TopoTarget AS, Kobenhavn, Denmark), Belinostat (TopoTarget AS, Kobenhavn, Denmark), sulfonamide hydroxamic acid, mocetinostat (MethylGene), Entinostat (MS-275, Bayer AG), MG-2856 (MethylGene), MG-4230 (MethylGene), MG-4915 (MethylGene), MG-5026 (MethylGene), belinostat (TopoTarget AS), abexinostat (Celera), panobinostat (Novartis), CG-200745 (CrystalGenomi), SB-939 (S*BIO), chidamide (HUYA Bioscience), CHR-3996 (Chroma Therapeutics), AR-42 (Arno Therapeutics), RG-2833 (RepliGen), OCID-4681-S-01, (Orchid Pharmaceuticals), PCI-34051 (Pharmalcyclics), DAC-60 (Genextra), KAR-2581 (Karus Therapeutics), resminostat (Nycomed Pharma), 45C-202 (Nycomed Pharma), YM-753 (Astellas), ACY-1216 (Acetylon), KAR-3000 (Karus Therapeutics), CU-906 (Curis), IKH-02 (IkerChem), ACY-257 (Acetylon), HDAC3 inhibitors (RepliGen), KAR-3166 (Karus Therapeutics), ONCO-101 (Oncoholdings), GSK424887 (GlaxoSmithKline), AGO178 (Novartis), TC5214 (AstraZeneca), pharmaceutically acceptable salts thereof, and combinations thereof.

14. The method according to claim 13, wherein the HDACi is SAHA, VPA, pharmaceutically acceptable salts thereof, or combinations thereof.

15. The method according to claim 10, wherein the SSRI is selected from the group consisting of fluvoxamine, trazodone, indeloxazine, viloxazine, dapoxetine, duloxetin, fluoxetine, olanzapine, tramadol, paroxetine, tramadol, paroxetine mesylate, venlafaxine, citalopram, escitalopram, demexiptiline, vilazodone, nitroxazepine, desvenlafaxine, sertraline, venlafaxine, milnacipran, minaprine, quinupramine, amine transporter inhibitor (AMRI), venlafaxine (Auspex), DSP-1053 (Dainippon Sumitomo Pharma), SEP-228432 (Dainippon Sumitomo Pharma), DA-8031 (DA-8031), escitalopram (Lundbeck), NSD-788 (Neurosearch), SKL-10406 (SK Holdings), BMS-820836 (AMRI), pipamperone, doxepin (Winston Pharmaceuticals), levomilnacipran, clonazepam (Zydus) pharmaceutically acceptable salts thereof, and combinations thereof.

16. The method according to claim 15, wherein the SSRI is fluoxetine, pharmaceutically acceptable salts thereof, or combinations thereof.

17. A composition for treating or ameliorating the effects of a mood disorder comprising an effective amount of an SSRI, an HDACi, and a pharmaceutically acceptable carrier.

18. The method according to claim 1, wherein the patient has been chronically stressed.

19. The method according to claim 1, wherein the patient has a history of early life stress.

20. The method according to claim 1, wherein the HDACi selectively inhibits HDAC 1, 3, 8, and combinations thereof.

21. A method for preventing, treating or ameliorating the effects of age-related cognitive deficits in a patient with a history of early life stress comprising administering to the patient in need thereof an effective amount of an HDACi.

22. A composition for preventing, treating or ameliorating the effects of age-related cognitive deficits in a patient with a history of early life stress comprising an effective amount of an HDACi and a pharmaceutically acceptable carrier.

23. A method for identifying and treating a patient population that suffers from a mood disorder that is more likely to respond to a combination SSRI/HDACi treatment comprising:
(a) treating a patient population with an SSRI;
(b) determining which patients in the patient population are not responding to or who have low responsiveness to the SSRI treatment in step (a); and
(c) to those patients determined to be not responding to or to have low responsiveness to the SSRI treatment, administering the SSRI and an HDACi in an amount effective to treat the mood disorder.

24. The method according to claim 2, wherein the HDACi is Entinostat (MS-275, Bayer AG) or a pharmaceutically acceptable salt thereof.

25. The method according to claim 10, wherein the HDACi is Entinostat (MS-275, Bayer AG) or a pharmaceutically acceptable salt thereof.


SEQUENCE LISTING (18 sequences)

SEQ ID NO: 1 - DNA, 20 bp, Artificial
  Synthetic Construct
cacgccaagtgtgtggagtt

SEQ ID NO: 2 - DNA, 20 bp, Artificial
  Synthetic Construct
catcagcatgggcaagttga

SEQ ID NO: 3 - DNA, 21 bp, Artificial
  Synthetic Construct
gctgcgactggcttcaactac

SEQ ID NO: 4 - DNA, 20 bp, Artificial
  Synthetic Construct
gctgcttggcttcactaggc

SEQ ID NO: 5 - DNA, 18 bp, Artificial
  Synthetic Construct
gtgatcgattaggctgct

SEQ ID NO: 6 - DNA, 20 bp, Artificial
  Synthetic Construct
attccccatgtcctcgaatg

SEQ ID NO: 7 - DNA, 21 bp, Artificial
  Synthetic Construct
cctcgagaatgtgatcaggga

SEQ ID NO: 8 - DNA, 20 bp, Artificial
  Synthetic Construct
ggccttgacgtttgagagca

SEQ ID NO: 9 - DNA, 21 bp, Artificial
  Synthetic Construct
ggaggactgcattcaggtcaa

SEQ ID NO: 10 - DNA, 20 bp, Artificial
  Synthetic Construct
tcatcaggaccactctcgcc

SEQ ID NO: 11 - DNA, 20 bp, Artificial
  Synthetic Construct
ctgggcaggtagtcaggtcc

SEQ ID NO: 12 - DNA, 19 bp, Artificial
  Synthetic Construct
ttgggatagccgtcagggt

SEQ ID NO: 13 - DNA, 20 bp, Artificial
  Synthetic Construct
gtgcctgattgacgggaagt

SEQ ID NO: 14 - DNA, 21 bp, Artificial
  Synthetic Construct
ccaccctccagaccagttgat

SEQ ID NO: 15 - DNA, 20 bp, Artificial
  Synthetic Construct
tcagctgagagcaggctgtg

SEQ ID NO: 16 - DNA, 19 bp, Artificial
  Synthetic Construct
tcagaagggctgacggttg

SEQ ID NO: 17 - DNA, 20 bp, Artificial
  Synthetic Construct
tggagggtttctgagcctca

SEQ ID NO: 18 - DNA, 20 bp, Artificial
  Synthetic Construct
ccataggccaagggcagtac